(19) United States
(12) Patent Application Publication
(10) Pub. No.: US 2026/0144989 A1
(43) Pub. Date: May 28, 2026

(54) METHOD AND APPARATUS FOR TRANSDERMAL STIMULATION TO TREAT OVERACTIVE BLADDER AND OTHER MEDICAL AILMENTS

(71) Applicant: EMKinetics, Inc., San Francisco, CA (US)

(72) Inventors: Daniel R. BURNETT, San Francisco, CA (US); Kondapavulur VENKATESWARA-RAO, San Jose, CA (US)

(73) Assignee: EMKinetics, Inc., San Francisco, CA (US)

(21) Appl. No.: 19/254,924

(22) Filed: Jun. 30, 2025

Related U.S. Application Data
(63) Continuation of application No. PCT/US2024/021658, filed on Mar. 27, 2024.
(60) Provisional application No. 63/454,785, filed on Mar. 27, 2023.

(51) Int. Cl. A61N 1/36

(52) U.S. Cl. CPC A61N 1/36031; A61N 1/36007

(57) ABSTRACT

Method and apparatus for transdermal stimulation to treat overactive bladder and other medical ailments are described herein. The method may generally comprise non-invasively positioning a first portion of a patient's body relative to an electrical stimulator such that a target nerve within the first portion of the body is in proximity to the electrical stimulator, delivering an electrical stimulus from the electrical stimulator to the target nerve, detecting electrical conduction through a response nerve or detecting a muscular response caused by an electrical conduction through the response nerve via at least one sensor positioned along a second portion of the body, receiving a signal from the at least one sensor indicative of the detected electrical conduction or muscular response thereby providing feedback about the existence of neuropathic-irresponsiveness along the target nerve, and adjusting stimulation parameters of the electrical stimulator based on the feedback.

### CROSS-REFERENCE TO RELATED APPLICATIONS

This application is a continuation of International Application No. PCT/US2024/021658 filed Mar. 27, 2024, which claims priority to U.S. Provisional Application No. 63/454,785 filed Mar. 27, 2023, each of which is herein incorporated by reference to the same extent as if each such individual publication or patent application were specifically and individually indicated to be so incorporated by reference.

### FIELD OF THE INVENTION

The present apparatus and methods relate generally to energy emitting apparatus and methods for providing a medical therapy. The apparatus and methods may provide for central and peripheral nerve and other tissue modulation or stimulation therapies

### BACKGROUND OF THE INVENTION

The overactive bladder (OAB) and urinary incontinence (UI) market in the United States is well over a $12 billion a year industry. It affects over 16% of all Americans, for a total U.S. market of approximately 34 million men and women each year. Due to social stigmas attached to OAB and UI, as well as misunderstanding of the signs and symptoms associated with OAB and UI, only 40% of those affected (13.6 M) seek treatment. Of those 13.6 million individuals, nearly 30% are unsatisfied with their current therapy.

Current treatment options for OAB and UI are exercise and behavioral modifications, pharmacological therapies, surgical intervention, and neuromodulation. Although each of these treatment options targets the UI and OAB populations, each has severe limitations.

Exercise and behavioral modifications often require patients to adhere to stringent routines, including scheduled voiding, maintenance of a bladder diary, and intense exercise regiments. While this may be a viable option for a small group of highly dedicated individuals, its daily impact on one's life makes it an unattractive option for most individuals.

Pharmacological intervention is the most widely prescribed therapy for OAB and UI. Unfortunately, as with the ingestion of any chemical, patients are often subject to side effects from their drug therapy. This is especially detrimental in older and elderly patient populations where interaction with other prescribed medications can have adverse effects. Further, there is a high rate of dissatisfaction, approximately 30%, amongst individuals using pharmacological treatment.

Surgical intervention is an extremely invasive treatment and often results in the long-term, and in some cases permanent, requirement for catheterization. The high expense of these procedures, coupled with the negative impact the procedures have on the patients quality of life, make this an option only when all other treatment options have been exhausted.

Neuromodulation is another treatment alternative for OAB and UI patients. Sacral nerve stimulation (SNS) has shown itself to be an effective treatment option for those with OAB or UI. However, the procedure requires the permanent implantation of an electrical stimulation device in the patient. One estimate puts the cost at nearly $14,000 with additional routine care costs of $593 per patient per year. Additionally, SNS's risk of battery failure, implant infection, and electrode migration, lead to a high reoperation rate and make this procedure unattractive.

### SUMMARY

In certain variations, a method for providing transdermal electrical stimulation therapy to a patient is provided. The method may include positioning a stimulator electrode over a glabrous skin surface overlying a target nerve of a subject. Electrical stimulation may be delivered through or across the glabrous skin surface to the target nerve to stimulate the target nerve, while remaining safe and tolerable to the patient. Electrical stimulation may be delivered at frequencies that may be painful or intolerable when applied over non-glabrous surfaces of the body. The electrical stimulation may be utilized to treat various conditions, e.g., urinary incontinence and overactive bladder.

Various types of electrodes may be utilized as a stimulator electrode, e.g., a surface electrode, microneedle electrode, a TENS (transcutaneous electrical nerve stimulation) patch or other electrode that may be positioned over or on the skin surface. Optionally, a conductive substance may be injected or implanted near the target nerve to improve electrical conductivity to the target nerve from the stimulatory electrode. Electrical stimulation may be delivered intermittently or on a chronic basis and may include one or more electrical signals designed to be constructive and/or destructive in order to improve tissue penetration and/or signal tolerance.

In certain variations, nerve stimulation resulting from the electrical stimulation therapy may be detected via at least one sensor positioned on or near the subject. The sensor may provide a signal indicative of the detected electrical stimulation thereby providing feedback about the efficacy of the applied electrical stimulation therapy such that the therapy may be adjusted or optimized. The feedback loop may be queried such that the electrical stimulation therapy may be adjusted to ensure that the minimum amount of energy is being applied to stimulate the target nerve while reducing the risk of burns or intolerance. Optionally, the feedback loop may be queried such that the positioning of the stimulator electrode may be adjusted to optimize the electrical stimulation therapy. Various sensors may be utilized, including but not limited to a surface electrode, a microneedle electrode, or motion sensor. In certain variations, the sensor may detect conduction of motor and/or sensory nerves. For example, a sensor may detect afferent or efferent nerve stimulation of the target nerve or other nerve or related nerve.

In certain variations, a ground electrode may be utilized. For example, the ground electrode may be positioned on the subject to facilitate penetration of an electrical current from the stimulatory electrode through the glabrous surface to stimulate the target nerve.

In certain variations, a stimulator electrode may be positioned over or attached to a glabrous skin surface with an adhesive or other form of attachment or fastener. In other variations, a stimulator electrode may be positioned over or held in contact with a glabrous skin surface with an ergonomic applicator.

Various applicators for positioning electrodes over various regions of the body to deliver electrical stimulation, e.g., transdermal electrical stimulation, may be utilized. For example, an applicator may be in the form of an insole configured to be positioned against, over, or in contact with the plantar surface or a glabrous surface of the foot such that an electrode positioned on the insole may deliver electrical stimulation through or across the glabrous skin surface, to a target nerve or other tissue within the foot. The insole may be configured to be positioned in an orthotic or a shoe such that electrical stimulation may be delivered to the subject while the subject is walking. In other variations, an applicator may be a foot plate or foot rest or cradle or mat on which a foot or other portion of the leg or body may be positioned to receive electrical stimulation through or across a glabrous skin surface, from the plate, rest or cradle or mat or from electrodes of the plate, rest or cradle or mat.

In one method of performing electrical stimulation therapy, the method may generally comprise non-invasively positioning a first portion of a patient's body relative to an electrical stimulator such that a target nerve within the first portion of the body is in proximity to the electrical stimulator, delivering an electrical stimulus from the electrical stimulator to the target nerve, detecting electrical conduction through a response nerve or detecting a muscular response caused by an electrical conduction through the response nerve via at least one sensor positioned along a second portion of the body, receiving a signal from the at least one sensor indicative of the detected electrical conduction or muscular response thereby providing feedback about the existence of neuropathic-irresponsiveness along the target nerve, and adjusting stimulation parameters of the electrical stimulator based on the feedback.

In another aspect of the method, the target nerve and the response nerve are the same.

In another aspect of the method, the first portion of the body and the second portion of the body are the same.

In another aspect of the method, the method may further comprise categorizing a degree of the neuropathic-irresponsiveness into at least one stage of neuropathy.

In another aspect of the method, the at least one stage of neuropathy comprises stage one, which is characterized by numbness and/or pain, stage two, which is characterized by constant pain, stage three, which is characterized by intense pain, or stage four, which is characterized by complete numbness and/or loss of sensation.

In another aspect of the method, receiving the signal from the at least one sensor comprises sensing F-wave signals from the detected electrical conduction or muscular response, where the F-wave signals are indicative of the existence of neuropathic-irresponsiveness along the target nerve.

In another aspect of the method, adjusting stimulation parameters of the electrical stimulator comprises adjusting a modulation of the electrical stimulus.

In another aspect of the method, adjusting stimulation parameters of the electrical stimulator comprises adjusting a frequency of the electrical stimulus.

In another aspect of the method, adjusting stimulation parameters of the electrical stimulator comprises adjusting a waveform of the electrical stimulus.

In another aspect of the method, adjusting stimulation parameters of the electrical stimulator comprises adjusting at least one of modulation, frequency, or waveform of the electrical stimulus.

In another aspect of the method, adjusting stimulation parameters of the electrical stimulator comprises adjusting at least one of modulation, frequency, and waveform of the electrical stimulus.

In another aspect of the method, adjusting stimulation parameters of the electrical stimulator comprises adjusting a position of the electrical stimulator relative to the first portion of the body.

In another aspect of the method, adjusting stimulation parameters of the electrical stimulator comprises adjusting at least one of modulation, frequency, waveform, and positioning of the electrical stimulator.

In another aspect of the method, the method may further comprise determining a location of neuropathic-irresponsiveness along the response nerve.

In another aspect of the method, determining the location of neuropathic-irresponsiveness comprises delivering the stimulus or receiving the signal via an array of electrodes configured for placement against the first portion of the body.

In another aspect of the method, delivering the electrical stimulus comprises delivering the stimulus or delivering the electrical stimulation via an array of electrodes spatially configured for placement against the first portion of the body.

In another aspect of the method, the array of electrodes is configured to deliver the electrical stimulation intermittently to determine a location for positioning of the electrical stimulator relative to the first portion of the body.

In another aspect of the method, providing feedback about the existence of neuropathic-irresponsiveness comprises comparing the signal from the at least one sensor against a range of values indicative of neuropathic-irresponsiveness.

In another aspect of the method, the range of values is obtained from a patient population.

In another aspect of the method, the range of values is obtained from the patient.

In another aspect of the method, the method may further comprise applying the electrical stimulation therapy to mitigate incontinence in the patient.

In another aspect of the method, the method may further comprise applying the electrical stimulation therapy to mitigate over active bladder in the patient.

In another aspect of the method, the method may further comprise applying the electrical stimulation therapy to mitigate menstrual pains, to mitigate substance abuse, tremors, or Parkinsons in the patient.

In another aspect of the method, non-invasively positioning comprises positioning the first portion of the patient's body relative to the electrical stimulator such that a sciatic nerve, or a branch thereof, within the first portion of the body is in proximity to the electrical stimulator.

In another aspect of the method, non-invasively positioning comprises positioning the first portion of the patient's body relative to the electrical stimulator such that a tibial nerve, or a branch thereof, within the first portion of the body is in proximity to the electrical stimulator.

In another aspect of the method, non-invasively positioning comprises positioning the first portion of the patient's body relative to the electrical stimulator such that an ulnar nerve, or a branch thereof, within the first portion of the body is in proximity to the electrical stimulator.

In another aspect of the method, any of the steps above may be combined in any number of combinations and in any order and are intended to be within the scope of this disclosure.

In one variation of the apparatus for performing electrical stimulation therapy, the apparatus may generally comprise an electrical stimulator configured for non-invasive placement against a first portion of the body such that the electrical stimulator is positionable into proximity to a target nerve within the first portion, at least one sensor positioned along a second portion of the body, and a controller in electrical communication with the electrical stimulator and the at least one sensor, wherein the controller is configured to: deliver an electrical stimulus from the electrical stimulator to the target nerve, detect electrical conduction through the target nerve or detect a muscular response caused by an electrical conduction through the target nerve via at least one sensor positioned along the second portion of the body, receive a signal from the at least one sensor indicative of the detected electrical conduction or muscular response thereby providing feedback about the existence of neuropathic-irresponsiveness along the target nerve, and adjust stimulation parameters of the electrical stimulator based on the feedback.

In another aspect of the apparatus, the controller is further configured to categorize a degree of the neuropathic-irresponsiveness into at least one stage of neuropathy.

In another aspect of the apparatus, the at least one stage of neuropathy comprises stage one, which is characterized by numbness and/or pain, stage two, which is characterized by constant pain, stage three, which is characterized by intense pain, or stage four, which is characterized by complete numbness and/or loss of sensation.

In another aspect of the apparatus, the controller is further configured to sense F-wave signals from the detected electrical conduction or muscular response, where the F-wave signals are indicative of the existence of neuropathic-irresponsiveness along the target nerve.

In another aspect of the apparatus, the controller is further configured to adjust the stimulation parameters based on a detection of neuropathic-irresponsiveness along the target nerve.

In another aspect of the apparatus, the controller is further configured to modify electrical stimulation therapy to the target nerve based on the detection of neuropathic-irresponsiveness.

In another aspect of the apparatus, the controller is further configured to modify an amplitude of the electrical stimulus to overcome the neuropathic-irresponsiveness.

In another aspect of the apparatus, the controller is further configured to modify a waveform of the electrical stimulus to overcome the neuropathic-irresponsiveness.

In another aspect of the apparatus, the controller is further configured to modify a frequency of the electrical stimulus to overcome the neuropathic-irresponsiveness.

In another aspect of the apparatus, the apparatus may further comprise an array of electrodes in electrical communication with the electrical stimulator, wherein the array of electrodes is configured for non-invasive placement against the first portion of the body such that the array of electrodes is positionable into proximity to the target nerve within the first portion.

In another aspect of the apparatus, the controller is further configured to determine a location of neuropathic-irresponsiveness along the target nerve via the array of electrodes.

In another aspect of the apparatus, the controller is further configured to determine a location of the electrical stimulator to optimize detection of the electrical conduction or muscular response.

In another aspect of the apparatus, the controller is further configured to compare the signal from the at least one sensor against a range of values indicative of neuropathic-irresponsiveness.

In another aspect of the apparatus, the array of electrodes is configured for positioning into proximity of a sciatic, tibial, or ulnar nerve, or a branch thereof, within the first portion of the body.

In another aspect of the apparatus, any of the features described above may be combined in any number of combinations and are intended to be within the scope of this disclosure.

### BRIEF DESCRIPTION OF THE DRAWINGS

The drawings constitute a part of this specification and include exemplary embodiments of the invention, which may be embodied in various forms. It is to be understood that in some instances various aspects of the embodiments may be shown exaggerated or enlarged to facilitate an understanding of the embodiments.

FIG. 1 shows a view of the underside or glabrous surface of the foot and exemplary sites for delivering electrical stimulation.

FIG. 2 shows a perspective view of one variation of an insole for delivering electrical stimulation over a glabrous surface of the foot.

FIG. 3 shows a perspective view of one variation of electrodes for delivering electrical stimulation over a glabrous surface of a foot.

FIG. 4 shows a perspective view of one variation of a hand applicator for delivering electrical stimulation over a glabrous surface of a hand.

FIG. 5 shows a perspective view of one variation of electrodes for delivering electrical stimulation over a glabrous surface of a hand.

FIG. 6 shows an example of an electromyography (EMG) reading.

FIG. 7 shows branches of the Tibial nerve

FIGS. 8 and 9 shows electrode placement and innervation of the plantar portion of the foot.

FIG. 10 shows an exploded view of the external generator or controller that can be used in some embodiments of the present invention.

FIG. 11 shows the generator/controller in FIG. 10 in a non-exploded view.

FIG. 12 shows another embodiment of a generator/controller.

FIG. 13 shows four exemplary patch electrodes that are suitable to use with the present invention.

FIG. 14 shows electrodes designed for the palmar surface of the hands along with F/M wave detecting electrodes.

FIG. 15 shows a pulse train of one embodiment.

FIG. 16 shows an M-Wave and F-Wave display.

FIGS. 17A-C show an example of an electrode array.

FIGS. 17D-E show examples of an electrode array on a mat or platform.

FIGS. 18A and 18B show flow charts of the device's process for detecting and handling neuropathy.

FIG. 19 is a block diagram of a data processing system.

### DETAILED DESCRIPTION

Electrical stimulation may be directed to nerves underlying glabrous skin surfaces of the body (e.g., the palms and soles). Higher levels of power (than what would be utilized and tolerated on non-glabrous skin surfaces) may be utilized when delivering electrical stimulation transdermally, through or across a glabrous skin surface, while remaining safe and tolerable. For example, stimulation of the plantar nerves or other nerves of the foot via a surface electrode positioned over the glabrous surface of the plantar surface is highly tolerable and results in sensation similar to that found with needle-based, invasive stimulation of the posterior tibial nerve, but in a non-invasive manner.

Various devices or applicators may be applied to the feet and/or hand or other body portions in order to stimulate nerves underlying glabrous surfaces on an intermittent or continuous basis. Electrical stimulation may be delivered via surface or microneedle electrodes. Various devices may be used in conjunction with implantable or injectable substances in order to improve electrical conductivity to a target nerve. As described herein, the various methods and devices for providing transdermal electrical stimulation over a glabrous skin surface may be used to treat any disorder that is impacted by neuromodulation including, but not limited to:
overactive bladder, urinary incontinence, fecal incontinence, chronic pain, depression, migraine, epilepsy, obesity, restless leg, or foot drop. The methods may be applied either intermittently or, if necessary, on a chronic basis. A stimulator electrode surface may be held in contact with a glabrous surface via an adhesive or an ergonomic applicator.

Feedback may be provided to the stimulator to indicate that stimulation is occurring as intended. This may involve an EMG type measurement device, a motion sensor or other sensing device. This feedback loop may be intermittently queried so that the stimulation may be adjusted to ensure that the minimum effective amount of energy is being used to stimulate to reduce the risk of burns or intolerance.

In certain variations, the device may include an ergonomic wrap, platform or cradle. The sensor feedback may allow for optimization of the positioning of electrodes based on sensor feedback further reducing the risk of burns or intolerance while increasing the efficacy of the neuromodulation.

The methods and devices described herein may be utilized to stimulate various body tissues, including nerve, muscle, skin, vasculature, or any other organ or tissue within the human body. The methods and devices may be used to treat any suitable condition or perform any suitable function via neuromodulation regardless of whether the stimulation source is electromagnetic fields, direct electric current, ultrasound, or RF fields.

In certain variations, methods, systems and/or devices for performing neuromodulation and/or low frequency induction therapy through glabrous skin surfaces, e.g., through palmar or plantar surfaces, are provided. The methods and system may be utilized for treating or preventing various conditions, such as urinary incontinence (UI) and/or overactive bladder (OAB).

For example, a patient suffering from UI or OAB may place the glabrous surface of their foot over an insole, foot rest or foot plate applicator to provide contact between the glabrous surface of their foot and a stimulator electrode of the insole, foot rest, or foot plate applicator. Alternatively, a tethered or wired electrode may be used without an applicator or separate from an applicator. The electrode may be attached to or held in contact with the glabrous surface with an adhesive or as a cutaneous patch.

The stimulator electrode may be positioned over the glabrous surface along the course of a target nerve. The stimulator electrode may be positioned proximal to a stimulation site to ensure that afferent nerve stimulation occurs. A ground electrode may be placed on the body as well. One or more sensing electrodes may be placed along the path of the nerve conduction or motor or sensory impulse, or elsewhere. Stimulator and/or sensing electrodes may be connected or coupled to a pulse generator. The pulse generator and electrodes may be incorporated or integrated into an insole, foot plate, patch, mat or other applicator. Alternatively, the electrodes may be connected or coupled to a pulse generator where the electrodes are not incorporated or integrated into the insole, foot plate, patch, mat or applicator.

Electrical stimulation via the stimulator electrodes may begin at a low amplitude and may slowly be ramped up or increased until nerve conduction is detected by the sensor electrode and/or detected by the patient who may signal that motor conduction has occurred, e.g., by pressing a button or other indicator. This ramping may be performed manually, or automatically, i.e., by the controller.

Once stimulation is detected, the electrical pulses or electrical stimulation may continue for the directed duration of use or therapy. Electrical stimulation may be delivered intermittently to provide intermittent therapy. For example, electrical stimulation may be delivered for 15-30 minute intervals. In another example, electrical stimulation may be delivered continuously to provide continuous therapy.

A sensor may remain in place for the duration of the electrical stimulation therapy to ensure that effective stimulation occurs the entire time or substantially the entire time, and to allow for correction or adjustment if the signal deteriorates. A controller for operating a pulse generator or sensor may be powered by a portable power source (e.g., a battery) or a fixed power source (e.g., a traditional wall outlet).

In certain variations, the electrical stimulation may have of a square wave electric signal at a frequency of about 5 Hz to about 60 Hz at the targeted tissue depth. The square wave configuration of the signal may be generated via Fourier transformation or may be a ramped current generated in any manner.

The insole, foot rest, patch or foot plate may be removed from the body when therapeutic stimulation is not being delivered. The insole, foot rest, patch or foot plate may be reapplied, in some embodiments, along with a sensor patch (which may be disposable) as indicated. For example, the electrical stimulation therapy may be administered on a daily basis, where one or more of the above steps are repeated.

Electrical stimulation may be delivered according to any of the variations described herein, in a manner such that the stimulation provides motor, sensory and/or subthreshhold stimulation. Any of the various energy based stimulation systems described herein may be utilized to provide therapy in various settings, e.g., in home use or to provide ambulatory type therapies.

In certain variations, as described herein, a device, applicator, pad, bracelet, ring or garment e.g., a gel pad or gel pad electrode, may include one or more electrodes, or have one or more electrodes coupled thereto. The device applicator, pad, patch, bracelet, ring, garment and/or electrodes may deliver or provide energy, e.g., electrical, magnetic or electromagnetic, stimulation through or across a glabrous skin surface, e.g., a plantar or palmar skin surface. Pain receptors may be located deep under glabrous skin surfaces (e.g., located on the foot or on the palm) allowing the patient to have a higher tolerance to pain resulting from stimulation of these surfaces, than they might have at another area of their body. Applying stimulation over these surfaces allows for stimulation to be delivered at a higher frequency or power or at a greater strength or for a longer duration because of the higher tolerance to pain that a patient exhibits at these surfaces or areas.

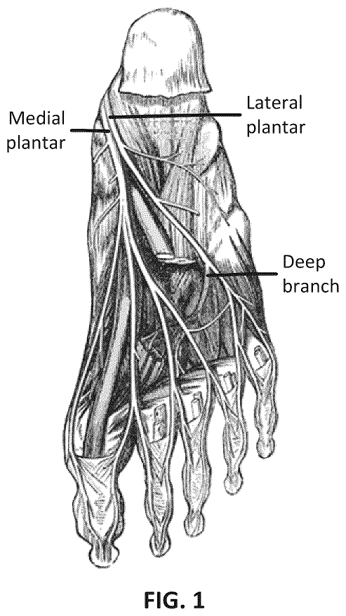

FIG. 1 shows the anatomy of a foot, providing a view of the underside, sole or plantar or glabrous surface of the foot. The medial and lateral plantar nerves innervate the sole of the foot and arise from the posterior branch of the tibial nerve. Various stimulator sites for stimulating the plantar nerves or branches thereof may be located near, in proximity to or along the plantar nerves.

In certain variations, electrical stimulation may be delivered transdermally, through or across a glabrous surface on a subject, e.g., through or across a glabrous surface of the foot or hand. The electrical stimulation may be sufficient to generate conduction of motor and/or sensory nerves. An electrical stimulation may be delivered by placing one or more electrodes anywhere over or on a glabrous skin surface overlying one or more target nerves or other target tissue. Stimulator and/or ground electrodes may be utilized. Exemplary electrodes may include but are not limited to surface electrodes, dry electrodes, gel electrodes, microneedle electrodes or any other suitable electrode for delivering an electrical stimulus. An electrode may be adhered or otherwise attached to a glabrous surface. Optionally, an electrode may be held or positioned in contact with, in proximity to, or over a glabrous surface with a wearable garment, cradle, applicator or body portion rest or support (as described in further detail herein).

Delivery of electrical stimulation through or across a glabrous surface of the body via an electrode positioned over a glabrous surface, e.g., a glabrous surface on a palmar or plantar surface, unexpectedly allows for the use of a higher frequency and/or higher amplitude electrical pulsation or electrical stimulus to deliver the electrical stimulation than would otherwise be safe and/or tolerable to deliver electrical stimulation through a non-glabrous surface of the body. For example, an electrical stimulus having a frequency of about 5 Hz to about 60 Hz (a range found to be effective for generating motor and/or sensory nerve conduction of the posterior tibial nerve) may be utilized to stimulate a target nerve (to generate motor and/or sensory nerve conduction therein) or tissue through or across a glabrous skin surface (via an electrode positioned over the glabrous skin surface) in a manner that remains safe and tolerable to the patient and avoids burns or injury. Optionally, an electrical stimulus having a frequency of about 5 Hz to about 60 Hz, or greater than about 15 Hz, may be utilized.

It is contemplated that other energy sources, for example, an electromagnetic or magnetic stimulus having a frequency of about 5 Hz to about 60 Hz, or greater than about 15 Hz, may be utilized to simulate a target nerve or tissue through a glabrous surface in a manner that remains safe and tolerable to the patient.

Various electrodes and/or applicators for applying an electrical or other energy based stimulation to a patient are described herein.

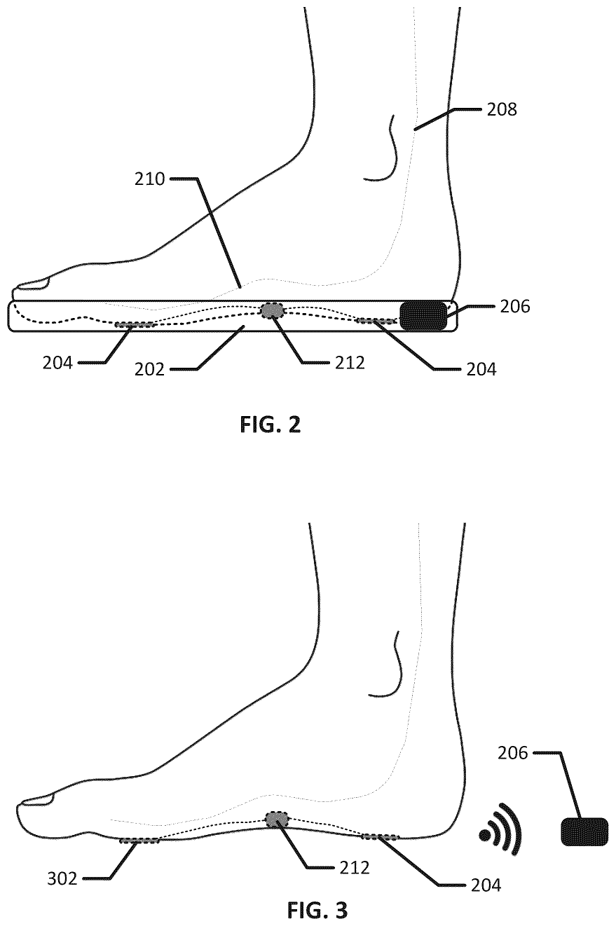

FIG. 2 shows one variation of an ergonomic insole or shoe applicator for delivering electrical stimulation over the glabrous surface of a foot. The ergonomic insole 202 may include one or more stimulator electrodes 204 for delivering electrical stimulation to a user. The electrode 204 may be attached to or positioned in the ergonomic insole 202. The ergonomic insole 202 may hold or position the stimulator electrode 204 against, in contact with, or in proximity to a glabrous surface of the foot, to deliver the electrical stimulation through the glabrous surface to an underlying target nerve or tissue. For example, electrical stimulation may be directed at a branch of tibial nerve 208, such as plantar nerve 210. Optionally, the electrodes may be utilized for providing stimulation, sensing and/or grounding. In certain variations, the electrode may be attached to one or more wires or may be wireless and/or coupled to an electrical pulse generator or other generator or controller and/or may not be attached to or positioned in an ergonomic insole. Any of the electrodes described herein may be durable, reusable, and/or disposable.

A controller 206 including an electrical pulse generator or other generator or controller may be coupled to one or more electrodes 204. The electrical pulse generator may be incorporated into or attached to ergonomic insole 202. A ground electrode or other ground may optionally be provided. A ground electrode (not shown) may be attached to a strap or band extending from or attached to the insole, an orthotic, shoe, or shoe applicator, sock or elsewhere on the user's body. The ground electrode may be attached to one or more wires or may be wireless and/or may be coupled to a stimulator electrode and/or the pulse generator or controller. The ground electrode may be positioned anywhere on the foot. For example, the ground electrode may be positioned over a posterior or upper surface of the foot to encourage or facilitate deeper penetration of the electrical current or stimulation, through the glabrous surface of the foot, to a plantar nerve or other target nerve within the foot. Alternatively, the ground electrode may be positioned on the glabrous surface of the foot.

In certain variations, the electrical pulse generator or controller may be located distant or remotely from an ergonomic insole and the electrode positioned therein. For example, the electrical pulse generator may attached elsewhere on the body, e.g., attached to a belt, inside a pocket or strapped to the calf or other region of the body. The pulse generator may communicate with or be coupled to the stimulator electrode or other electrodes via a wire or wirelessly.

In certain variations, the ergonomic insole may be custom built into an orthotic, shoe applicator or other support for the foot. For example, the ergonomic insole may be built into an orthotic or shoe applicator providing the user with the freedom to walk around while receiving electrical stimulation therapy. The mechanical energy created by walking may be used to power the controller.

In other variations, an applicator may be in the form of a foot or hand plate, cradle or support. For example, a user may rest or position their bare foot on a foot plate to receive electrical stimulation therapy from the plate or from one or more electrodes attached to the plate. In another variation, a user may rest or position the palm of their hand on a hand plate or support to receive electrical stimulation therapy from the plate or from one or more electrodes attached to the plate to provide upper extremity stimulation.

The ergonomic insole may include one or more sensor electrodes. For example, in one variation, a stimulator electrode 204 may also act as a sensor electrode. In some embodiments, an additional sensor electrode 212 may be provided. The sensing electrode may detect stimulation of the underlying target nerve, to provide feedback regarding the efficacy of the applied electrical stimulation therapy. For example, the sensing electrode may detect motor and/or sensory nerve conduction. Detection of and feedback regarding nerve stimulation via the sensing electrode may provide for automatic adjustment of the treatment parameters or may guide or allow for manual adjustment of the treatment parameters in order to optimize stimulation therapy.

In certain variations, one or more separate or dedicated sensor electrodes may be attached to or positioned in the ergonomic insole to continuously and/or intermittently sense the stimulation of a nerve. The ergonomic insole may hold or position the sensor electrode against, in contact with, or in proximity to a glabrous surface of the foot, to detect stimulation of the underlying target nerve, to provide feedback regarding the efficacy of the applied electrical stimulation therapy. For example, the sensor electrode may detect motor and/or sensory nerve conduction. Detection of and feedback regarding nerve stimulation via the sensor electrode may provide for automatic adjustment of the treatment parameters or may guide or allow for manual adjustment of the treatment parameters in order to optimize stimulation therapy. The sensor electrode may be coupled or connected to the electrical pulse generator/controller. In certain variations, the sensor electrode may be attached to one or more wires, may be wireless and/or may be coupled to a pulse generator and/or may not be attached to or positioned in an ergonomic insole.

A sensor or applicator with a sensor may be capable of sensing stimulation of a nerve underlying a glabrous surface allowing for manual or automatic feedback to adjust the parameters of the stimulation or the position of the area being stimulated in order to optimize the stimulator therapy.

In certain variation, a sensor electrode may be positioned or placed along the path of the nerve conduction or motor or sensory impulse. Optionally, the sensor electrode may be positioned proximal to a stimulator electrode, along the nerve conduction path

In certain variations, the electrodes may be attached to one or more wires, may be wireless and/or may be coupled to an electrical pulse generator or other generator or controller and/or may not be positioned in an applicator, patch, multiple patches, or support. For example, one or more stimulator electrodes may be adhered to or otherwise attached to a glabrous skin surface of a patient. One or more ground electrodes and/or one or more sensor electrodes may also be adhered to or otherwise attached to the patient. The stimulator electrode, sensor electrode and/or ground may be coupled to an electrical pulse generator/controller and/or each other using wires or wirelessly. The electrical pulse generator/controller may be positioned in various locations or located anywhere on a patient. For example, the electrical pulse generator or controller may be held by a patient, located on a belt or strap worn by the patient, or positioned in a pocket or pouch on the patient.

For example, FIG. 3 shows one variation of a stimulator electrode 204 attached to a glabrous skin surface of the foot or sole of the foot. A ground electrode, or 2^{nd} stimulator electrode 302 may be attached to the foot at another location. The stimulator and/or ground electrodes may be coupled, in a wired or wireless fashion, to electrical pulse generator/controller 206, which may be positioned at various locations on or away from the patient. The electrodes/sensors may be coupled to a wireless transmitter/receiver to transmit/receive signals to/from the controller. Optionally, sensor electrode 212 may be attached to the patient to detect nerve or other tissue stimulation and to provide feedback regarding the efficacy of the therapy to optimize the therapy. The sensor may be coupled to the electrical pulse generator 206 in a wired or wireless connection.

In certain variations, systems and methods for providing electrostimulation(ES) of the posterior tibial nerve in individuals with OAB and UI, via transdermal electrical fields, are provided. Various electrodes for delivering the electrical stimulation may be utilized, including but not limited to surface electrodes with and without gel, microneedle electrodes, and electrodes under strong pressure.

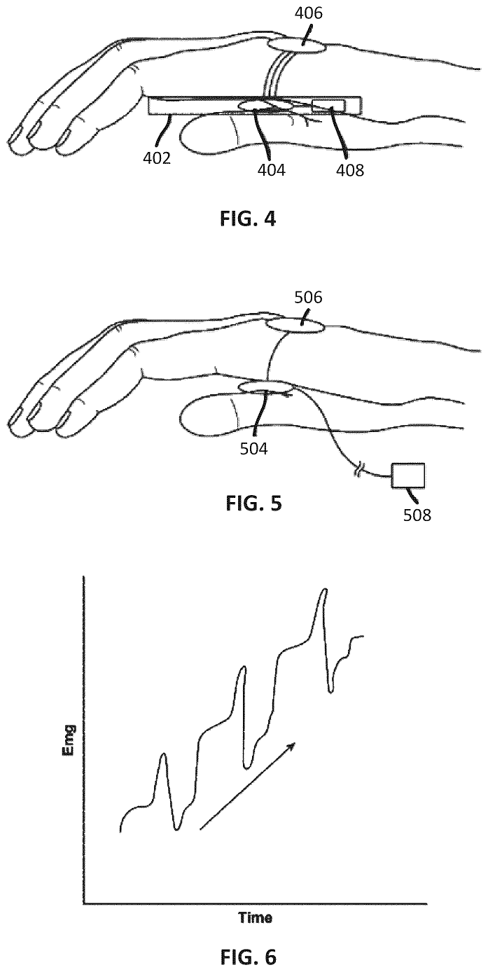

Other ergonomic applicators, designed for other regions of the body, may be utilized for delivering electrical stimulation over various regions of the body. For example, FIG. 4 shows a glove, brace or other hand wrap or glove like applicator 402. The applicator 402 may include one or more stimulator electrodes 404, such that the applicator 402 may hold or position an electrode 404 over or in contact with a glabrous surface of the hand or palmar region of the hand to deliver electrical stimulation through or across the glabrous surface, to an underlying target nerve or tissue within the hand. A ground electrode 406 or other ground may be attached to the hand at another location and/or may be attached to a strap of the applicator. The ground electrode may be on the back or palm of the hand. The stimulator and/or ground electrodes 404, 406 may be coupled to an electrical pulse generator/controller 408, which may be positioned in the applicator 402 or at various locations on or away from the patient. Optionally, a sensor electrode (not shown) may be attached to the patient to detect nerve or other tissue stimulation and to provide feedback regarding the efficacy of the therapy in order to optimize the therapy. The sensor may be coupled to the electrical pulse generator 408.

FIG. 5 shows another variation of a stimulator electrode 504 attached, via adhesive for example, to a glabrous skin surface of the hand or palm of the hand. A ground electrode 506 may be attached to the hand at another location. The stimulator and/or ground electrodes 504, 506 may be coupled to an electrical pulse generator/controller 508, which may be positioned at various locations on or away from the patient. Optionally, a sensor electrode (not shown) may be attached to the patient to detect nerve or other tissue stimulation and to provide feedback regarding the efficacy of the therapy to optimize the therapy. The sensor may be coupled to the electrical pulse generator/controller 508.

More than one stimulator electrode may be present in any of the embodiments disclosed herein. Any of the embodiments disclosed herein may or may not include a ground electrode and/or a sensor electrode.

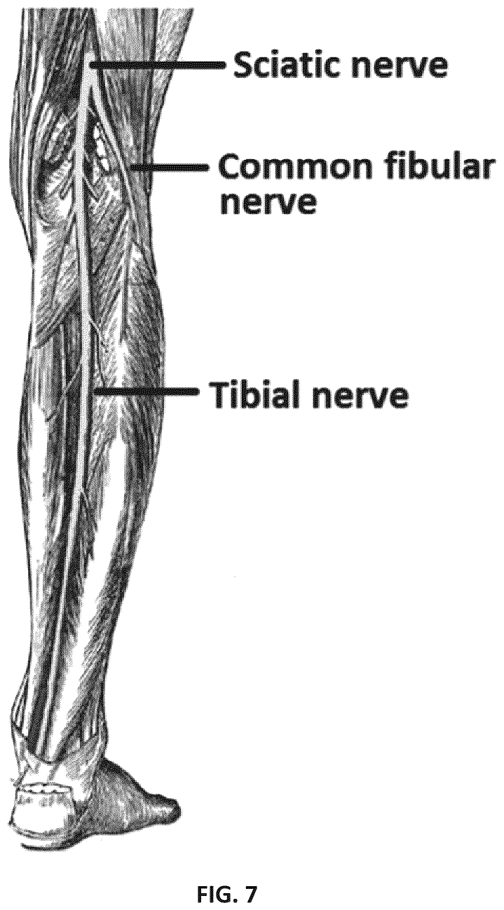

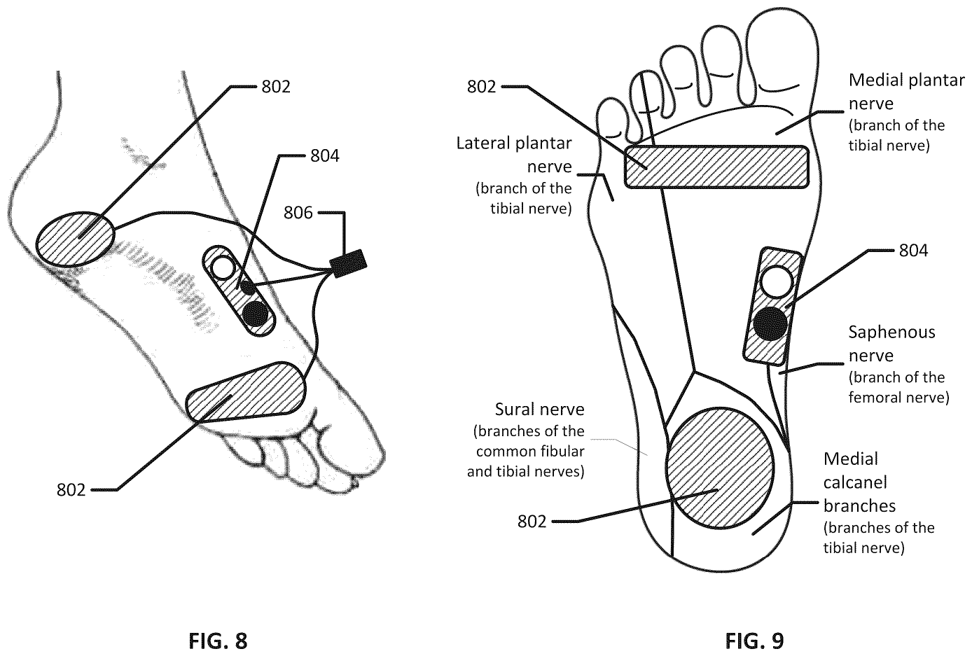

FIGS. 7-9 show branches of the Tibial nerve, electrode placement and innervation of the plantar portion of the foot. In FIG. 7, the Tibial nerve and its branches are shown extending from the Sciatic nerve. The Tibial nerve continues to the bottom of the foot as show in FIG. 1. FIG. 8 shows possible placement of electrodes 802 and EMG pad 804 containing multi electrodes, all of which are connected to controller 806. Both electrodes 802 may be microneedles or both may be patch or surface electrodes or one may be a microneedle electrode while the other is a patch electrode. FIG. 9 shows how placement and size/shape of the electrodes can correspond to the various cutaneous innervation regions on the bottom of the foot.

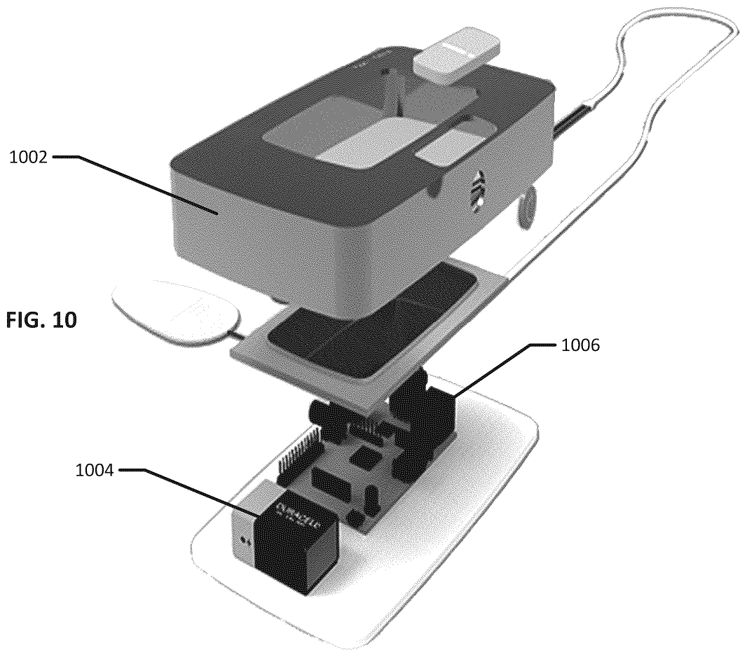

FIG. 10 shows an exploded view of the external generator or controller that can be used in some embodiments of the present invention. The generator/controller has housing 1002 which contains internal components. The internal components may include a microprocessor, memory, battery 1004, on/off switches and various other electronic components 1006 for storing various operational and functional programs and executing those programs in accordance with a desired instruction set for the relevant indication or neurological malady. Such programs will include but not be limited to instructions related to the parameters for electronic pulses which emit from the electrodes. They typical instruction set will include the desired pulse width, frequency, amplitude and the desired time intervals, and possibly, time length, for the therapy. The instructions may include more or less information as desired, such as burst modes, high frequency sequencing, coordinated reset functions, treatment and physical response history, and the like. In addition to delivering pulses, the generator/controller may also contain electronics for sensing F and M waves, and the ability to display, interpret and/or act on such sensed F and M waves. “Acting on” may mean that the controller analyzes and determines whether the parameter instruction set requires adjustments in order to maximize therapy effectiveness. If an adjustment is required, the instruction set will either make an automatic adjustment or send an alert or other information to the display to prompt a manual adjustment.

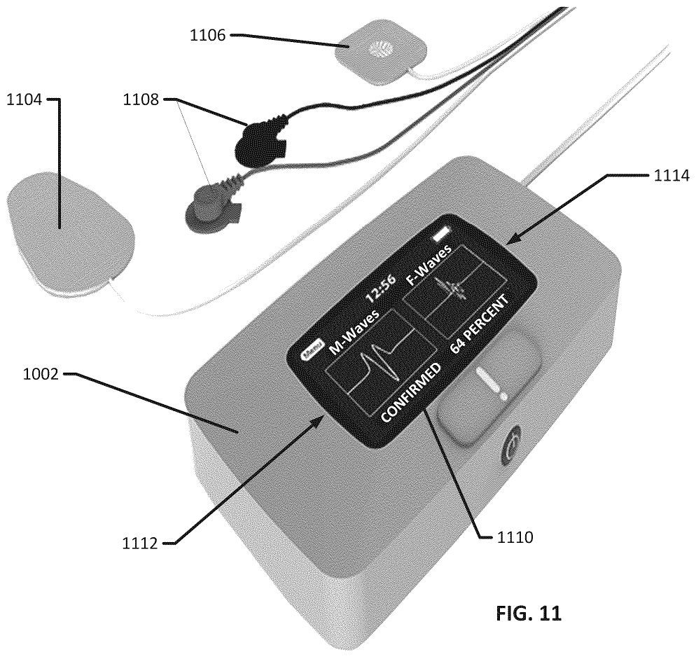

FIG. 11 shows the controller in FIG. 10 in a non-exploded view with electrodes 1104 and 1106 and F/M wave sensors 1108, all connected to the controller via insulated wires. The display 1110 shown in this example displays an M-wave feedback confirmation result 1112 and an F-wave display 1114 that shows the F-wave percent. The display may show other information such as the pulse parameters described above. In some embodiments, the controller provides a manual mode switch for determining which mode the system will be set to. It also contains an on/off switch and a display for displaying various information relating to the therapy and/or pulse parameters. The system may include two electrodes, one of which can be a stimulating electrode and one a return electrode. Any of the systems disclosed herein may contain two surface, or skin, electrodes or two percutaneous microneedle electrodes or one microneedle electrode and one surface electrode. This embodiment may also have one or more EMG sensing electrodes to sense F-Wave and M-Wave electrodes. All the electrodes may be connected to a generator/controller via a connection cable or wireless connection.

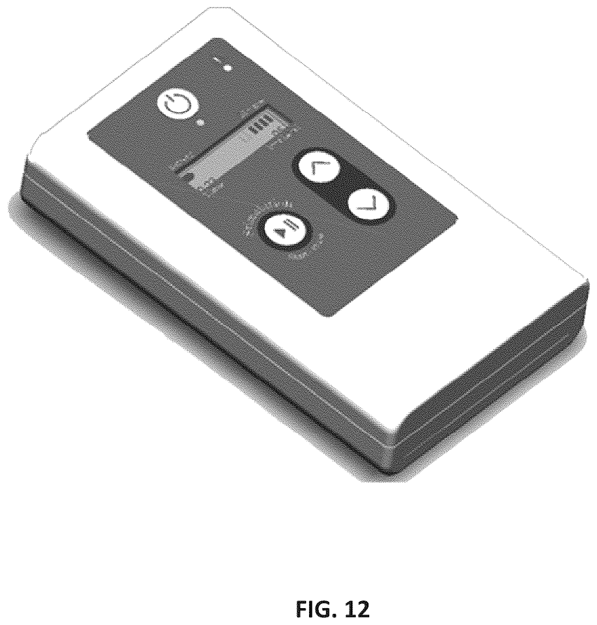

FIG. 12 shows another embodiment of a generator/controller. This generator/controller may have all, some of, or additional attributes as those generator/controllers disclosed herein. FIG. 12 has the components in a compact, portable case. It may have advanced features, such as wireless communication, such as Bluetooth, or other wireless technology, and the ability to communicate with current smartphones or other computers via different applications. For example, any of the embodiments disclosed herein may include a controller which sends, and possible receives, data to/from a centralized server wirelessly. The connection may be via wi-fi, Bluetooth, cellular connection etc. The controller may be a smartphone, tablet, or other computer and/or may communicate with a smartphone, tablet, or other computer. The data sent from the controller and/or received by the controller may be anonymous and/or patient specific data. The data sent/received may be specific to one person or may be aggregate data of more than one user.

Alternatively, all or some of the functions of the generator/controller may be incorporated into a mobile computing device, such as a smart phone, tablet, mobile phone etc. The mobile computing device may include an “app” or application installed on the mobile device, which controls the functions of the electrodes, display and/or mobile device. The electrodes may be incorporated into the casing of the generator/controller, for example in a ball or handheld format. The electrodes may be incorporated into a casing which is physically attached to a mobile computing device. The casing may be powered by the mobile computing device or may be powered separately. Electrodes may be dry and built into the case for stimulation and EMG so that the user is in contact with the electrodes when the case is gripped. Electrodes may alternatively be wet and replaceable with attachments, such as snaps, which attach to, or connect to, the case. The app may use an EMG incorporated into the case, or may use accelerometers in the mobile device itself to measure intensity of stimulation, by sensing physical response to the stimulation. Since the gripping of the case may cause the fingers of the user to lay on the touchscreen display, the touchscreen may sense contraction of the fingers/hand and give an indication of the body's response to stimulation presence/intensity. The touchscreen may sense the manner in which the fingers move (distance, direction, number of fingers etc.) or with 3D touch, the pressure on the screen may be measured.

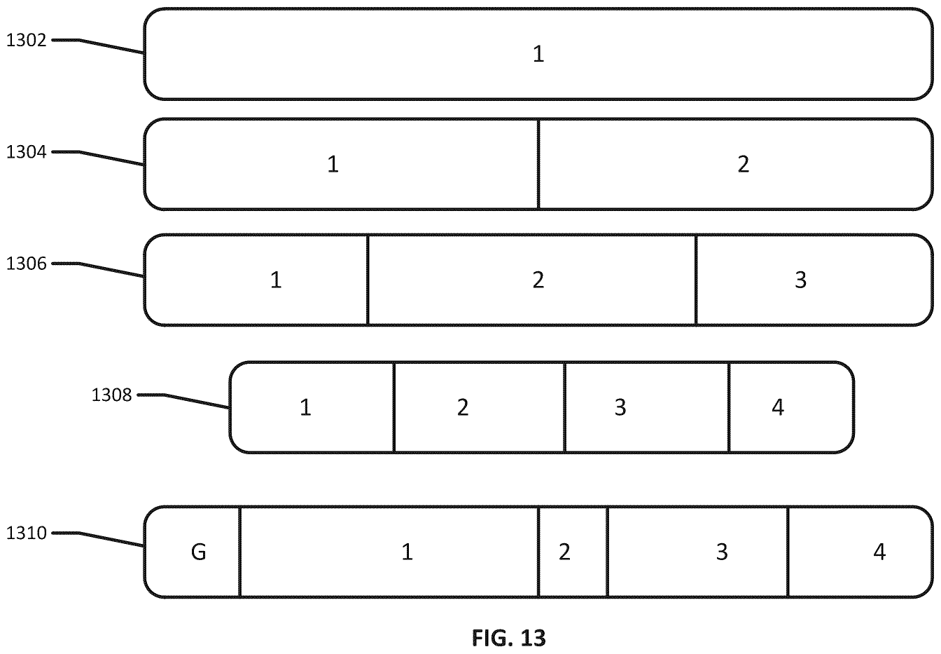

FIG. 13 shows four exemplary patch electrodes that are suitable to use with the present invention. Patch 1302 is a patch electrode with only one surface electrode contact area, 1. Patch 1304 is a patch electrode configuration where the patch contains 2 electrode contact areas, 1 and 2. Likewise, patch 1306 includes 3 electrode contact areas, 1, 2 and 3. Patch 1308 includes 4 contact areas, 1, 2, 3 and 4. Patch 1310 includes 5 electrode contact areas, with one contact area, G, being configured to serve as a possible ground for the other electrodes.

FIG. 13 shows a few of the different configurations of electrodes. These contact areas can be equally spaced or spaced according to the anatomy of the desired stimulation area. For example, in patch 1308, contact area 4 may be the same size or smaller or larger than contact area 3 depending on the stimulation area desired. Additionally, FIG. 13 show patch electrodes, but it should be understood that the descriptions here are translatable to microneedle electrodes used alone or in conjunction with patch electrodes or fully implantable electrodes or both. The relative shape and/or area of the different electrodes may vary. For example, the shapes may be rectangles or irregularly shaped.

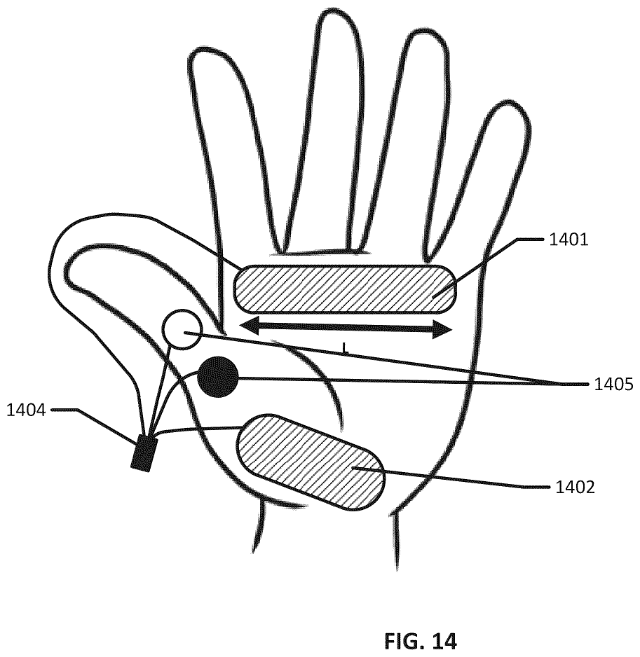

FIG. 14 shows electrodes designed for the palmar surface of the hands along with F/M wave detecting electrodes 1405. In FIG. 14, contact electrode(s) 1401 are placed across the upper palm portion of the hand and second electrode 1402 is place across towards, near or on the heel of the hand. The electrodes are connected to generator/controller 1404 and electrical pulses are delivered to the hand per the instructions on the controller. Depending on the length “L” and placement of electrode 1401, electrical stimulation of the hand will stimulate the Median or the Ulnar nerve or both. It should be understood that depending on the therapy and other considerations, the desired stimulation may be either one or both the Ulnar or Median nerve or elsewhere on the body.

The present invention contemplates placing the electrode patches in one or more of the combinations listed herein, or in other combinations, as required to achieve the desired stimulation therapy. Also, the present invention contemplates using single patch electrodes or one or more patches with two or more electrodes each that may stimulate with a more refined or focused field. For example, it is known in the art to use “anodal guarding” to localize the electric field to stimulate the desired areas while electrical “guarding” or reducing the field to not stimulate those other areas surrounding the particular focus of the stimulation therapy. Additionally, the invention contemplates that sensing electrode(s) may be used in these combinations. Several figures show patch electrodes, but it should be understood that the descriptions here are translatable to microneedle electrodes used alone or in conjunction with patch electrodes or fully implantable electrodes or both.

The type of energy delivered to the nerve or nerves during stimulation by the device may depend on the pulse parameters. For example, in one therapy type, the pulse parameters have a frequency of somewhere between about 1-20 Hz or 1-30 Hz, a pulse width between about 0 -500 microseconds and an amplitude of between about 0 and 90 mA. In another therapy, high frequency stimulation can be used, namely frequencies from about 20 hz up to about 10,000 hz. Such high frequencies can be effective using either external electrodes, or fully implantable electrodes, or both. The electrodes are placed in a location to stimulate the Ulnar nerve, the Median nerve or both nerves.

The energy pulse amplitude may also vary during the treatment. This modulation can increase both comfort and efficacy. For example, the pulse energy applied to the electrodes may be at 100% amplitude for X pulses (for example, 5 pulses) and then at a lower amplitude (for example, 50% amplitude) for Y pulses (for example, 5 pulses). The pulse amplitude and length may vary, for example, there may be two amplitude levels, or 3 or more. The different amplitude durations may be the same, or they may be different. The amplitude variations may be preset, or may be reset during the use of the device.

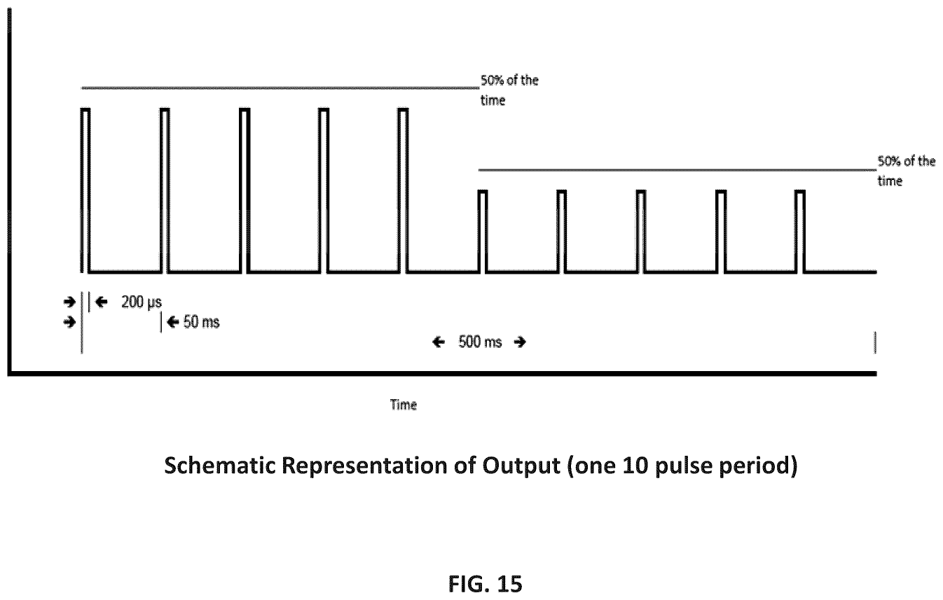

FIG. 15 shows one example of varying amplitudes where the amplitude magnitude is 100% and 50%, each for 5 pulses, alternating between 100% and 50% amplitude. More complicated variations are also envisioned. For example, the amplitude may vary in a sine function, or a multiple level function with the different amplitude lengths varying per amplitude. The amplitude variations may be the same across users or custom to each user, or even each user experience.

The parameters of the amplitude function include amplitude as a percent of maximum, amplitude time length, amplitude number of pulses, number of different amplitudes, order of different amplitudes, etc. These parameters may be communicated to a remote server for analysis to optimize treatment of individual users in the future. The data may also be aggregated (preferably anonymously) to develop treatment algorithms for different patient populations. Effectiveness input from the individual users may also be incorporated into the algorithms, for example pain level over time, comfort level, medication taken etc.

Similarly, pulse frequency parameters can also be modulated. Similarly, pulse width parameters can also be modulated.

In some embodiments of the device, an amplitude (and/or frequency) modulator adapter may be added to a commercially available TENS (Transcutaneous Electrical Nerve Stimulation) device. This adapter would modulate the amplitude and/or frequency of the signal produced by the TENS device so that the signal delivered to the user via the electrodes can be optimized for comfort and efficacy. Sensors, such as F-wave and/or M-wave sensors, may also be added/incorporated.

Some embodiments allow the user to use the device inconspicuously, for example at work, or in a meeting. The electrodes may be part of a phone case so that the user's hand is in contact with the electrodes while holding the phone. The electrodes may be part of a beverage mug, computer mouse, steering wheel, etc.

### Principles of Electromyography

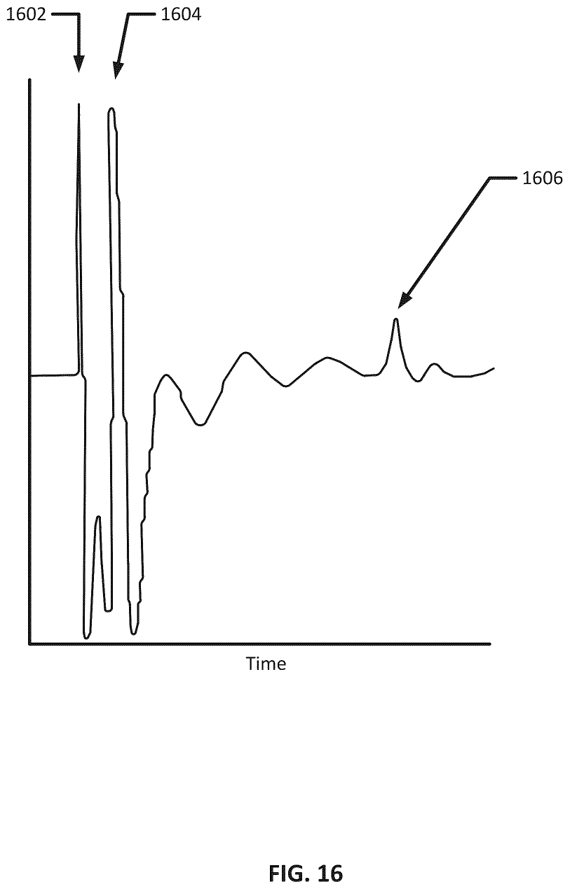

See FIG. 16 for the following discussion. The system may also capture and display electromyographic information during the treatment. Electromyography is the study of the electrical signals in the muscles for analysis of the behavior of the muscle or nervous systems in a body. During electrical neural muscle stimulation, signals called M-waves and F-waves are created by the body in the stimulated muscles and nerves in response to the electrical stimulation. In neuroscience, the M-Wave is the first, and the F-wave is the second, voltage change observed after electrical stimulation is applied to the distal region of a nerve. F-waves are often used to measure nerve conduction velocity, and are particularly useful for evaluating conduction problems in the proximal region of nerves (i.e., portions of nerves near the spinal cord). The signal is called the F-wave because it was initially recorded in the foot muscles.

In a typical F-wave study, a strong electrical stimulus (above maximal muscle stimulation levels) is applied to the skin surface above (proximally to) the distal portion of a nerve so that the impulse travels both distally (towards the muscle fiber) and proximally (back to the motor neurons of the spinal cord; these directions are also known as orthodromic and antidromic, respectively). When the orthodromic stimulus reaches the muscle fiber, it elicits a strong M-response indicative of muscle contraction (M-wave). When the antidromic stimulus reaches the motor neuron cell bodies, a small portion of the motor neurons fire in the other direction and an orthodromic wave travels back down the nerve towards the muscle. This reflected stimulus evokes a small proportion of the muscle fibers causing a small, second compound muscle action potential (CMAP) called the F-wave.

FIG. 16 shows a graph of detected voltage vs. time and shows M-wave 1604 and F-wave 1606 following stimulation 1602. This type of graph may be displayed by the controller to the end user. Alternatively, a different parameter of an M-wave and/or an F-wave may be displayed to the user, such as whether or not the wave exists, the amplitude of the wave, the travel time of the wave, etc. Any of the parameters may be used by the controller in a feedback loop to control any parameter of the stimulation.

The generator/controller may capture and display both the M-wave and F-wave during confirmation and treatment modes. Being able to view the M-waves and F-waves during treatments may allow the operator to confirm adequate stimulation of the nerve in question (Ulnar, Median or Tibial) and its branches in the feet; and the median and Ulnar nerves, and/or their branches, in the hand.

In certain variations, transdermal electrical stimulation therapy may be provided to a patient or subject. One or more stimulator electrodes may be positioned over a glabrous skin surface overlying a target nerve of the patient. Electrical stimulation or an electrical stimulus may then be delivered via the stimulator electrode through or across the glabrous skin surface to the target nerve in the patient, to stimulate the target nerve. The electrical stimulation may be delivered at a level and in a manner sufficient to generate motor and/or sensory nerve conduction. For example, the electrical stimulation may be delivered at a frequency of about 5 Hz to about 80 Hz or about 20 Hz to about 60 Hz or greater than about 15 Hz, while remaining safe and tolerable to the patient. The stimulation may be delivered in a non-invasive manner.

The stimulator electrode may be positioned over or on various regions of the body. In certain variations, the stimulator electrode may be positioned over a palm or plantar skin surface. For example, in certain variations the stimulator electrode may be positioned over a glabrous skin surface overlying a plantar nerve of the foot, where the electrical stimulation may stimulate the plantar nerve. Stimulation of the plantar nerve may result in stimulation of the tibial nerve to treat various conditions, such as urinary incontinence or overactive bladder.

In some variations, an applicator may be a glove, brace or other hand wrap which is configured to be positioned against, in contact with, or over the palmar surface or a glabrous surface of a hand. An electrode of the glove, brace or wrap may deliver electrical stimulation through or across the glabrous skin surface to a target nerve or tissue within the hand.

Various applicators or ergonomic applicators may be utilized to provide transdermal electrical stimulation therapy to a subject. In certain variations, an applicator may include one or more stimulator electrodes and connect to and one or more electrical pulse generators or controllers. The electrical pulse generator may be coupled to the stimulator electrode. The electrical pulse generator may be incorporated into the applicator. Optionally, the electrical pulse generator may be separate from the applicator or located remotely from the applicator or stimulator electrode. An applicator may include one or more sensor electrodes configured to detect nerve stimulation and/or provide feedback about the efficacy of the applied electrical stimulation therapy. Such feedback may allow the therapy to be adjusted, modulated and/or optimized. A sensor may detect motor and/or sensory nerve stimulation. For example, a sensor may detect afferent or efferent nerve stimulation. In certain variations, the positioning of the stimulator electrode may be adjusted based on feedback from the sensor in order to optimize the electrical stimulation therapy.

The applicator may also include one or more ground or ground electrodes. The ground electrode may be a component of the applicator or may be attached to the subject separately via a strap or other attachment. The ground electrode may be positioned on the subject at a location that facilitates penetration of an electrical current through a glabrous surface to stimulate the target nerve, e.g., on the opposite surface of a body portion relative to the stimulator electrode.

In other variations, one or more stimulatory electrodes may be otherwise attached to a skin surface, e.g., as a wired or wireless patch, adhesive or microneedle electrode in the absence of an ergonomic applicator, to deliver electrical stimulation across or through a glabrous skin surface to stimulate an underlying target nerve or tissue. Such electrodes may be used in combination with one or more ground or sensor electrodes. Such stimulatory electrodes may deliver electrical stimulation or an electrical stimulus at various frequencies, e.g., at a frequency of about 5 Hz to about 60 Hz, or greater than about 15 Hz, while remaining safe and tolerable to the subject.

In certain variations, methods, systems and/or applicators for providing an energy based stimulation therapy to a subject are provided. An energy emitting device may be positioned in proximity to a glabrous surface overlying a target tissue. Energy may be delivered from the energy emitting device through or across the glabrous skin surface to the target tissue to stimulate the target tissue, such as a target nerve. Various energy sources or forms of energy may be delivered by the energy emitting device, including but not limited to, an electric current, an electromagnetic or magnetic field or electromagnetic or magnetic induction stimulation, ultrasound, or RF fields. Energy may be delivered at various frequencies. For example, the energy may be delivered at a frequency of about 5 Hz to about 60 Hz, or greater than about 15 Hz, while remaining safe and tolerable to the subject.

The energy based stimulation therapy may be utilized to treat various conditions, including but not limited to, overactive bladder, urinary incontinence, fecal incontinence, chronic pain, depression, migraine, epilepsy, obesity, restless leg syndrome, or foot drop. Energy may be delivered through a glabrous skin surface to provide neuromodulation to stimulate other tissue. Tissues that may be stimulated include but are not limited a central nerve, peripheral nerve, muscle, skin, or vasculature. Optionally, conductive substance may be implanted or injected near a target tissue to improve conductivity to the target tissue.

In one variation, a method for treating urinary incontinence or overactive bladder in a subject may include one or more of the following steps, a stimulator electrode, (e.g., in an applicator or as an adhesive or attachment electrode as described above) may be positioned over a glabrous skin surface overlying a plantar nerve or other nerve in the foot. An electrical stimulation may then be delivered through or across the glabrous skin surface to the plantar nerve to stimulate the plantar nerve which results in stimulation of the tibial nerve to treat urinary incontinence or overactive bladder, e.g., via stimulation of the sacral plexus or pudendal nerve.

In other variations, any of the various electrical stimulation methods and electrical stimulator applicators or electrodes may be utilized to treat any of the conditions described herein.

In certain variations, methods, systems and/or applicators for providing an energy based stimulation therapy to a subject are provided. An energy emitting device may be positioned in proximity to or over a skin surface overlying a target nerve or tissue. The energy emitting device may include any of the devices, systems or applicators described herein and/or illustrated in any of the various figures, e.g., an electrode or applicator for delivering electrical stimulation or an applicator for providing electromagnetic or magnetic stimulation or induction therapy. Energy may be delivered at a frequency of about 1 Hz to about 30 Hz through or across a skin surface (e.g., a glabrous skin surface or any other skin surface or non-glabrous skin surface) to the a target nerve to generate motor and/or sensory nerve conduction while remaining safe and tolerable to the subject. In certain variations, energy may be delivered at a frequency of less than 10 Hz to generate motor and/or sensory nerve conduction.

Energy delivered transdermally, through, or across a patient's skin at a frequency from about 1 Hz to about 30 Hz, or at a frequency of less than 10 Hz has unexpectedly been found to stimulate or generate motor and/or sensory conduction in a target nerve. For example, energy delivered transdermally, through, or across a patients skin at about 1 Hz to about 30 Hz, or at less than 10 Hz has unexpectedly been found to stimulate or generate motor and/or sensory nerve conduction of a tibial nerve, where such level of stimulation may be sufficient to treat a patient suffering from urinary incontinence, overactive bladder, fecal incontinence or other conditions. The energy may be delivered through or across a glabrous skin surface or non-glabrous skin surface or any other skin surface (e.g., any skin surface overlying a tibial nerve).

Various energy sources or forms of energy may be delivered by the energy emitting device, including but not limited to, an electric current, an electromagnetic or magnetic field, ultrasound, or RF fields. The energy based stimulation therapy may be utilized to treat various conditions, including but not limited to, overactive bladder, urinary incontinence, fecal incontinence, chronic pain, depression, migraine, epilepsy, obesity, restless leg syndrome, or foot drop. Energy may be delivered through a glabrous skin surface or any other skin surface to provide neuromodulation to stimulate other tissue. Tissues that may be stimulated include but are not limited a central nerve, peripheral nerve, muscle, skin, or vasculature. Optionally, conductive substance may implanted or injected near a target tissue to improve conductivity to the target tissue. In addition, the device, applicator, bracelet, ring, pad and/or electrode may include, carry or be coated with one or more drugs, agents or therapeutic substances. Optionally, one or more electrodes may be utilized without a pad or applicator and the electrodes may have a drug or agent coating on a surface of the electrode. In certain variations, the applicator, pad and/or electrode may be coated with a pain relief drug, anesthetic or numbing agent. The pain relief drug, anesthetic or numbing agent may provide the patient with an increased or improved pain tolerance or may reduce or eliminate the sensation of pain, such that the energy, e.g., electrical stimulation, may be applied or delivered at a higher frequency, higher power, greater strength and/or longer duration. The drug or agent may coat a surface of the applicator, pad and/or electrode such that the drug or agent is located between the applicator, pad and/or electrode and the glabrous skin surface of the patient when the applicator, pad or electrode is applied to the skin, thereby applying the drug to the skin. The drug or agent may be provided in a slow release form or as a slow release drug or agent, such that the coating provides a slow release of the drug, agent or active ingredient into the patient. Any of the applicators, pads, insoles, gloves, wraps, braces, plates, platforms, electrodes and/or other energy delivery devices described herein for delivering stimulation, e.g., through or across a glabrous surface, may be coated with or include a drug as described above.

In certain variations, stimulation e.g., energy stimulation such as electrical, magnetic or electromagnetic stimulation, may be provided through a glabrous skin surface at parameters that overcome or avoid habituation of the targeted tissue to the stimulation. For example, stimulation may be provided, e.g., continuously, for 10 minutes, where the stimulation is automatically paused or stopped for about a 30 second interval every 10 minutes. This intermittent pausing or interruption of stimulation may prevent or overcome habituation of the target tissue, e.g., nerve, muscle or other tissue, to the delivered stimulation.

In one variation, the treatment parameters for stimulating a target nerve or other tissue through a glabrous skin surface, e.g., a plantar or palmar skin surface, may include the following: Apply electrical or magnetic stimulation for a duration of 30 minutes, pausing for about a 30 second interval after 10 minutes of stimulation and then again after another 10 minutes of stimulation. In other variations, the treatment parameters may vary, e.g., stimulation may be applied for an interval ranging from 0 to 100 minutes or greater or as necessary to treat symptoms. In certain variations, for example, stimulation may be applied or provided for 20, 40, 50 or 60 minutes. In certain variations, for example, the pause in stimulation may be for an interval ranging from 0 to 100 seconds, or 5 to 60 seconds or 20 to 40 seconds.

In other variations, stimulation at varying frequency may be applied or provided for varying times with total stimulation of 20, 40, 50 or 60 minutes. In certain variations, for example, the change in stimulation frequency may be for an interval ranging from 0 to 100 seconds, or 5 to 60 seconds or 20 to 40 seconds.

In another variation, a sensor (e.g., EMG sensor) may be utilized, which may detect signs of habituation, e.g., decreased stimulation or excitation of a target nerve, muscle or other tissue (or lack of any resulting stimulation) or a reduced response to stimulation. Through a feedback loop, detection of habituation or stimulation levels by the sensor, may automatically cause the stimulator to pause or stop delivery of stimulation to the target nerve or tissue for a predetermined interval or duration or until habituation is no longer detected. In any of the variations described herein, the stimulation may be paused automatically, manually or via other controls or via an electrical pulse generator or controller in a predetermined or preset manner and/or based on feedback provided by a sensor.

In certain variations, electrical energy or stimulation may be applied to glabrous skin surfaces through electrodes, e.g., conductive microneedle patches, needles, or other electrodes, or by utilizing any of the devices or applicators described herein. Various electrodes, devices and/or applicators for providing stimulation may be applied to plantar or palmar surfaces of a patient. For example, if applied to plantar surfaces, electrodes may be applied to one foot or both feet. In cases where the stimulation is applied to one foot, the other foot may be used as ground, i.e., stimulation may be delivered into one foot and out the other foot. Various conditions may be treated using the devices, applicators and/or electrodes and/or the various methods, therapies and treatment parameters described herein, e.g., urinary incontinence, overactive bladder, epilepsy, migraines, and depression, etc.

In certain variations, treatment of a patient using electrical stimulation may be monitored and/or adjusted based on the detection of feedback signals using primarily F-wave detection or based on the detection of F-waves or M-waves, e.g., in a foot or hand. For example, detection of an F-wave or F-wave feedback (e.g., via a sensor or electrode sensor) may be used in monitoring and/or to adjust the applied stimulation therapy. Optionally, stimulation may be ramped up until an F-wave is detected and then adjusted as necessary. F-wave detection measures nerve conduction velocity. F-wave detection may be used as opposed to H-reflex detection, which measures reflectory reaction from muscles. In other variations, optionally both F-wave and H-reflex detection may be used or solely H-reflex detection may be used. Stimulating to a sub-motor stimulation may be performed. In certain variations, F-waves, M-waves and/or H-reflex may be detected in the foot, hand or other body part.

Electrical energy may be applied or delivered using any of the devices, applicators or electrodes described herein at specified frequencies and/or parameters or using various duty cycles. In one example, electrical energy may be applied at a frequency of 5 Hz to 20 Hz (e.g., to provide supermaximal stimulation) in a train of 5 pulses followed by a ¼ second break, and then another train of 5 pulses. Up to 10 pulses per treatment cycle may be provided. The train of 10 pulses can be repeated as needed. In another example, a train of 10 pulses followed by a break and then another 10 pulses may applied, e.g., at a frequency of 5 Hz to 20 Hz or at about 20 Hz. The ¼ second break time may be varied as well as the number of pulses applied and the number of cycles. This pulsed treatment may be applied through one or more or several cycles. The pulsed treatment or stimulation may produce F-waves which may be detected. In certain variations, any of the treatment parameters as described herein may be applied where only half of the pulses are stimulatory, e.g., one or more cycles of pulses may be provided at 20 Hz, where only half of the pulses are stimulatory.

Certain frequencies, e.g., frequencies applied at greater than 20 Hz, may lead to a painful condition called tetany (i.e., painful, involuntary muscle cramping), which is an indication that the upper level for frequency has been reached in the patient. Tetany may be monitored using a sensor, e.g., an EMG sensor or Electromyography, to provide feedback regarding tetany. Tetany may be distinguished by EMG. FIG. 6 shows an example of an EMG reading showing a gradual increase in the detected level of stimulation or electrical activity, up to a level where tetany is likely experienced. Once the system detects the beginning of tetany or provides tetany feedback, the treatment frequency or power (of the stimulation delivered by an electrode or device) may be reduced, adjusted or halted automatically or manually. The frequency at which tetany is experienced may vary in different individuals.

In one variation, a treatment device or applicator for delivering energy, e.g., electrical energy, may include a platform with one or more microneedles, microneedle electrode, or other electrodes positioned upon the platform. Microneedles may vary in length to penetrate different layers of the skin, e.g., to penetrate a callus on the skin. A patient may place their foot upon the platform to receive treatment. Optionally, a separate strap having a feedback/monitoring electrode or electrode sensor may be wrapped over the foot to place or position a feedback/monitoring electrode on the patient. The strap and feedback electrode or electrode sensor may be coupled to the platform or be separate. In another variation, the stimulatory electrode may be positioned in a shoe, sock or glove of a patient. In certain variations, a TENS electrode or stimulator may be utilized.

Detection of stimulation, excitation, or conduction using a sensor may provide information to optimize or adjust the applied therapy. If no or reduced stimulation, excitation, electrical activity, response, F-waves, and/or EMG is detected during treatment, this may be indicative of a mis-positioned foot, palm or other body part relative to the one or more electrodes or the device or applicator.

In certain variations, electrical, electromagnetic, magnetic energy and/or other energy may be utilized to provide stimulation according to any of the systems and/or methods described above.

The present invention may use electrical stimulation of one or more of the Ulnar nerve, the Median nerve, the tibial nerve, or other nerves, either alone or simultaneously, separately or with other types of therapy to treat various neurological conditions, including neuropathies, migraine, pain, headaches, depression, epilepsy, post-traumatic stress disorder, overactive bladder, incontinence, subcutaneous fat, effects of stroke, memory enhancement, etc. In some embodiments, treatment can be combined with certain drugs to increase treatment effectiveness.

Devices used to stimulate one or more of the Ulnar, Median or Tibial nerves can be a completely external device, a fully implantable device or a device that comprises both external and internal components. The devices typically comprise a pulse generating component that provides an electrical wave form as output. The electrical pulse comprises certain typical parameters, such as pulse width, frequency, number of pulses and amplitude among other parameters. The choice of parameters may depend on the nerve, the location of the electrodes, the desired effect sought and the amount of time the therapy treatment occurs.

Payment for usage of the system may be controlled by an application in communication with the controller. For example, erasable programmable read-only memory (EPROM) may be used to track the number and duration of uses of the system. Further usage may be prohibited after paid duration and/or number of treatments have been consumed. Payment via the app (or outside the app) may be required to continue treatments.

In some embodiments, the system may be used for a short time (a time duration which is less than that of a treatment session) without paying, but may not complete a treatment until a payment is made.

In some embodiments, the system may be used for a single, or a few treatments, without paying, but may not complete further treatments until a payment is made.

In some embodiments, the system may check for proper placements of the electrodes without payment, but will not allow treatments until a payment is made.

In some embodiments, the system may check for an F-wave, M-wave, impedance or other parameter without payment, but may not complete further treatments until a payment is made.

In some embodiments, the sensing electrodes may be the same as the stimulation electrodes. In other words, separate sensing electrode(s) may not be necessary. The sensing function may be performed by the controller during the pauses between stimulation pulses, or in longer pauses in stimulation.

In some embodiments, the sensing electrodes may sense for feedback during pauses in the stimulation. These pauses may last for 50 ms to several seconds.

Sensed feedback may be in the form of F-waves, M-waves, sensory nerve action potential (SNAP).

In some embodiments, more than one electrode may be incorporated into an electrode patch. In some embodiments, sensor electrodes may be incorporated into stimulation electrode patches, such as the patches shown in FIG. 13.

In some embodiments, an array of electrodes may be used on the plantar surface of the palm or the foot to establish optimal electrode placement, size and/or stimulation. This type of arrangement may be used to establish which size/configuration of electrode system will be used for ongoing treatment. In other words, an array of electrodes may be used in a doctor's office to “fit” a different device that the user may take home and use for regular treatments. The “fitting” configuration of the device may test different electrode placements, sizes, as well as different stimulation durations, levels, patterns, number of electrodes, etc., to find the optimal treatment configuration for any given patient. Other tests which may be incorporated into a “fitting” configuration include the ability of a patient to achieve a feedback signal, such as an F-wave, M-wave or SNAP. Different electrode placements/sizes/stimulations may be tested using an electrode array. The electrode array may be 1 dimensional, 2 dimensional or 3 dimensional.

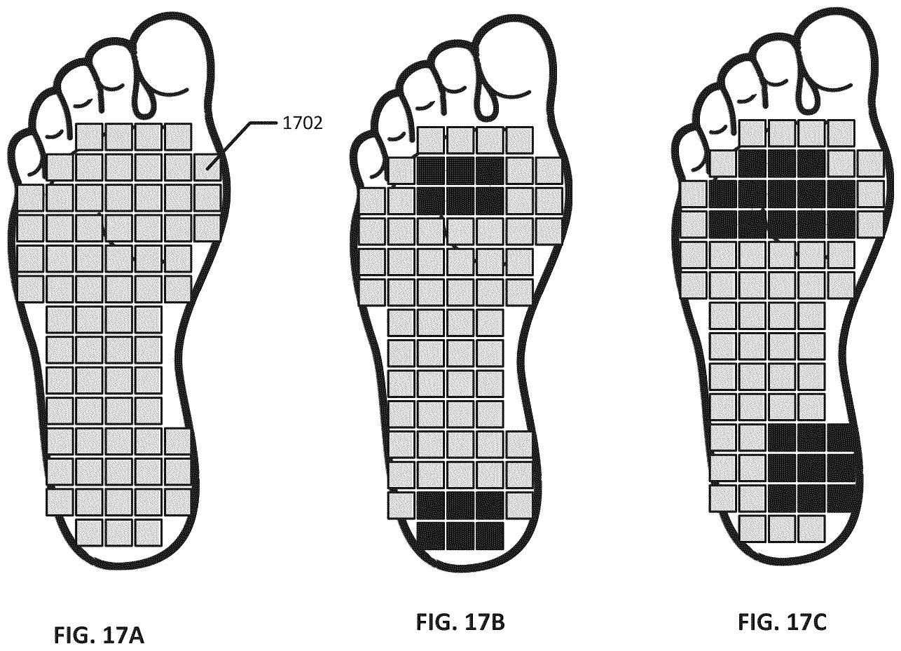

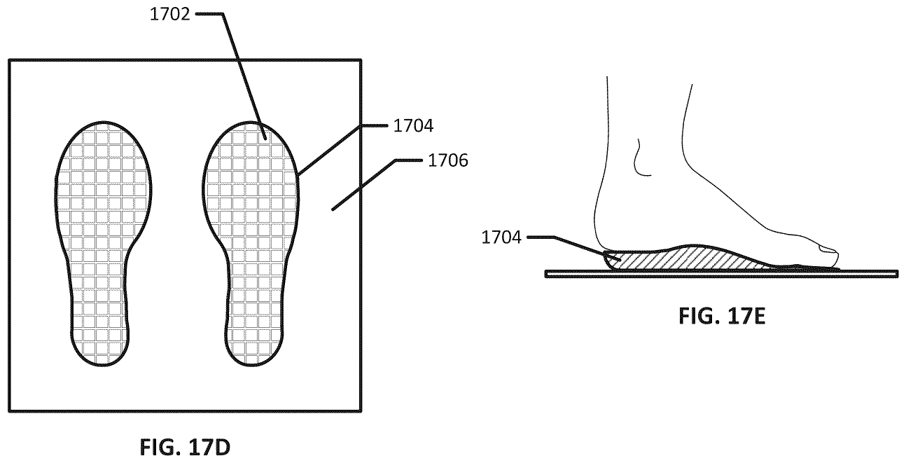

FIGS. 17A-C show an example of a 2 dimensional electrode array, which may be used in a “fitting” configuration of the system, or in a treatment configuration of the system. The electrode array includes several individual electrodes 1702. Different size, shape and stimulation sequences may be tested to determine the optimal treatment configuration. The optimal treatment configuration may be determined by the feedback signal, such as the F-wave, M-wave or SNAP signal, or by patient feedback or sensation, for example, patient sensation or patient twitching. Any of the electrodes 1702 in the array may be used as stimulation or sensor electrodes. The controller may automatically run through a test sequence to determine the optimal configuration or the tests may be run manually. The electrode array may be incorporated into an adhesive pad, an insole, or incorporated into a mat or station on which the user put his/her foot or palm. FIGS. 17B and 17C show two possible electrode result possibilities, where the darker electrodes represent the custom size and shape and location of optimal electrode configuration for different users.

Some embodiments include custom sizing/placement of electrodes. For example, a “fitting” configuration of the device may determine a particular electrode arrangement for optimal treatment of a given patient. The arrangement may be incorporated into an app and/or a custom application mat or pad, so that the user can duplicate the arrangement during treatments. The custom sizing and/or placement and/or stimulation pattern may be part of a prescription device for a patient. The device may include the ability to automatically manufacture a prescription or custom electrode array based on the fit/effectiveness/tolerance tests.

In some embodiments, the end user version of the device incorporates the “fitting” components. For example, the end user electrode pad may incorporate an array of electrodes similar to those shown in FIGS. 17A-17C. The end user controller may include the logic to run the fitting tests, or a subset of the fitting tests, either every time the user uses the product, or at certain intervals, or initially. The controller may automatically determine the optimal parameters of the electrode array based on feedback signals while running a series of tests which tests different parameters of the treatment and electrodes.

Some treatment patterns which may be tested and optimized include ramped stimulation (ramped up or down), periodic or paused stimulation, alternating stimulation levels, or different stimulation level patterns, different stimulation levels, different stimulation durations, etc.

Some electrode parameters which may be tested and optimized include electrode number, location, size, shape, and role (stimulating, sensing etc.).

The fitting test cycle may be different depending on certain patient demographics, such as age, size, sex, foot size, weight, etc. The fitting test cycle may include a series of steps. For example, the first test may be a simple stimulation to determine relative tolerance. Once that is determined, the controller may run another fitting test cycle that is appropriate for that patient's tolerance level. The fitting test cycle may have several layers so that the final fitting test cycle run may be a fairly quick test cycle. One of more of these test cycles may be re-run on the user's device during ongoing home use. For example, a user with small feet and a low tolerance for stimulation, may be sent home with a device programmed to run a different optimization test on the device than that of a user with larger feet and a higher tolerance for stimulation.

Some embodiments of the device may run a series of tests to determine the parameters which support minimum symptoms, for example overactive bladder and urge incontinence symptoms. Symptoms may be received manually, as through user input into an app, or may be received automatically, by monitoring urodynamic measurements.

Some embodiments of the device may run a test cycle or sequence to determine and/or maximize the effectiveness of the device. For example, some users may not produce F-waves or M-waves etc. throughout the test stimulation pattern range. The device may determine that these individuals are not good candidates for the device.

Some embodiments may test or maximize the effectiveness in other ways, for example, testing the ability of the device to curb symptoms, including overactive bladder and urge incontinence. The curbing of symptoms may be received manually, for example with a survey on an app, including patient comfort, satisfaction, symptom level etc., or may be automatically assessed. For example, automatic assessment may include the device integrating with devices which assess urodynamic measurements, such as bladder pressure before and/or after voiding, bladder volume before and/or after voiding, void volume, cystometry measurements, detrusor muscle contraction, void pressure, void flow rate, post void residual volume, maximum cystometric capacity, opening pressure, maximum detrusor pressure, urethral pressure profilometry, leak point pressure, video UDS, electromyography etc. The parameters may be monitored over time while using the device to assess effectiveness of the device. For example, if bladder pressure or bladder volume before voiding is increasing over time, this may be in indication that the device is effective in decreasing symptoms. For example, void volume or flow may be measured with a weight scale (weighing the voided urine), or a flow ring which may be separate from, or integrated with, a toilet seat. Another way to measure bladder volume or pressure is a strap around the abdomen with strain gauge or pressure sensor. Any of the urodynamic measurements may be measured by devices which are integrated with device embodiments disclosed herein.

Some embodiments of the device may require the user to drink a certain volume of water, and then run tests assessing and/or maximizing the treatment effectiveness over time.

Some embodiments of the device may collect 16 or more data points in a 30 day period to determine effectiveness of the treatment.

Some embodiments of the device may collect acute effectiveness and/or tolerance data. Some embodiments may collect effectiveness and/or tolerance data over time.

An electrode array incorporated into either a user device or a fitting device may be manufactured using printed conductive ink or other methods.

Some embodiments of the system incorporate electrodes into a mat which remains on the floor. The mat may include custom sizing, for example an outline of the user's foot or a receptacle which is sized to the user's foot, which incorporates electrodes in the proper place for that user's treatment. The electrodes may be incorporated into a disposable layer which can be replaced after each use or after a set number of uses.

Some embodiments of the device may be worn in an ongoing basis, such as an insole. The device may provide stimulation continually, or at timed intervals. The device may include pressure sensors to determine with the user is walking or standing and may halt stimulation during these times. Mobile embodiments, such as these, may include a bluetooth or other wireless transmitter/receiver as well as a battery. The controller may be a phone, with a bluetooth or other wireless transmitter/receiver, or other separate device. Alternately, the controller may be wired to the insole and strapped to the leg or ankle of the user, or integrated into a sock, or shoe of the user. Some embodiments may have an on-demand switch to start or increase stimulation during social situations where finding a bathroom isn't possible.

In some embodiments, the stimulation electrode is the same as the sensor electrode. The stimulation and sensing may be alternated during use.

FIGS. 17D and 17E show embodiments of a mat or platform which incorporates an array of electrodes either for fitting purposes or for use by the user for ongoing treatments. Individual electrodes may be arranged on a 2 dimensional or 3 dimensional foot receptacle 1704, which may be incorporated into mat or platform 1706. Receptacle 1704 may be 2 dimensional or 3 dimensional, as shown in FIG. 17E. The form fitting shape of 3 dimensional receptacle 1704 allows all or most of the electrodes in the electrode array to contact the bottom of the foot. This allows testing or “fitting” of all electrode types, including stimulating electrodes, sensing electrodes and ground electrodes, in a variety of shapes, positions and sizes on the foot.

In some embodiments, the controller is portable, either incorporated into a smart phone or other small handheld device, or incorporated into a device which is attached to the user's body, so that the user may walk around or move around during treatments.

In some embodiments, the energy emitting systems may integrate with social media apps, such as Facebook, WeChat, etc. In some embodiments, the controller is a mobile phone. In some embodiments, social media apps are integrated with the software on the controller and/or the stimulation device. Software applications integrated with the energy emitting system may include interfaces which allow the user to track usage, buy usage sessions, compare usage statistics with those of other users, compete with other users, or with oneself, by comparing usage minutes, success, F-wave, M-wave, or other feedback, to those of others and/or over time.

In some embodiments, a software app may be integrated with the device. The software app may reside on the device, on a controller of the device, on a smart phone, on a computer, tablet etc. in some embodiments, the software app may include the ability to share a user's achievements, history, successes etc. For example, a user may achieve certain rewards or levels based on adhering to minimal treatment times/day or other time period. A user may also receive rewards or levels based on achieving a recommended level of treatment, for example, achieving a stimulation level where M-waves or F-waves or other feedback are sensed by the system. These rewards and/or levels and/or goals may be shared with other users in a competitive manor.

Some embodiments may include an app with a distracting activity, such as a game, to distract the user from any discomfort during treatment. The game or distraction may be integrated with the treatment level. For example, a game may provide more features or a higher level of entertainment after sufficient treatment minutes or after F-waves of M-waves or SNAP are detected.

In some embodiments rewards are provided for adequate treatment times and/or treatment levels (possibly based on F-wave or M-wave or SNAP detection). These rewards may include free electrodes or other supplies, or free treatments, or the removal of advertisements from the app.

In some embodiments, other types of distractions may be incorporated. For example, an electrode, or other physically distracting device, may be placed elsewhere on the body to distract the user from any discomfort associated with the treatment stimulation. Music or sound may be used as a distraction.

In some embodiments, the stimulation may be slowly ramped over time as the user becomes accustomed to the stimulation. For example, a user's optimal treatment stimulation level may be 5, but when the user first starts using the system, the stimulation level may be 4, and may automatically ramp up to 5 over time. The system may also incorporate a relief button that lowers the treatment level a small amount when pressed. The stimulation level may then either stay at the lower level, or ramp up to the prescribed level over time.

Some embodiments of the system include accelerometers which can detect muscle contractions, such as foot contractions, and automatically adjust the stimulation level either to achieve the contractions, or to reduce the contractions. Impedance sensors may also be used to detect muscle contractions/flex.

In some instances, use of the system may be contraindicated in users with certain forms of, or a certain degree of, neuropathy. The controller may asses the level and/or type and/or existence of neuropathy using parameters of F-waves, M-waves, SNAP and/or H-reflex signals. The assessment of neuropathy may include determining the type of neuropathy, determining the location of neuropathy and/or determining the level of neuropathy.

For example, F-wave velocity may be used to assess neuropathy. For example, an F-wave velocity below a certain threshold may indicate neuropathy. Alternatively, or additionally, F-wave latency, F wave amplitude, F-wave shape, F-wave slope, F-wave persistence, F-wave chronodispersion (the difference between the maximal and minimal latencies), minimal F-wave latency, maximal F-wave latency, F-wave existence, and/or any parameter of M-waves, SNAP and/or H-reflex may be used to assess neuropathy.

The F-wave, M-wave, SNAP and/or H-reflex signal may be measured using a sensor electrode or EMG sensor. The neuropathy assessment may be performed using any of the system embodiments disclosed herein. The neuropathy assessment may be performed by the same device that is used for treatment, or it may be performed by a different device. If performed by the same device, the neuropathy assessment may be performed using the same electrode positioning as treatment, or it may be performed using a different electrode positioning.

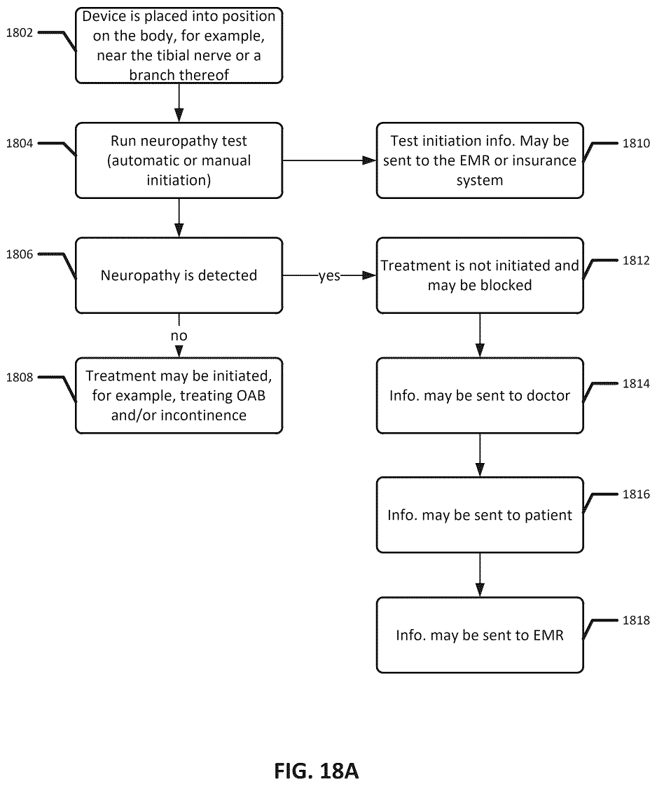

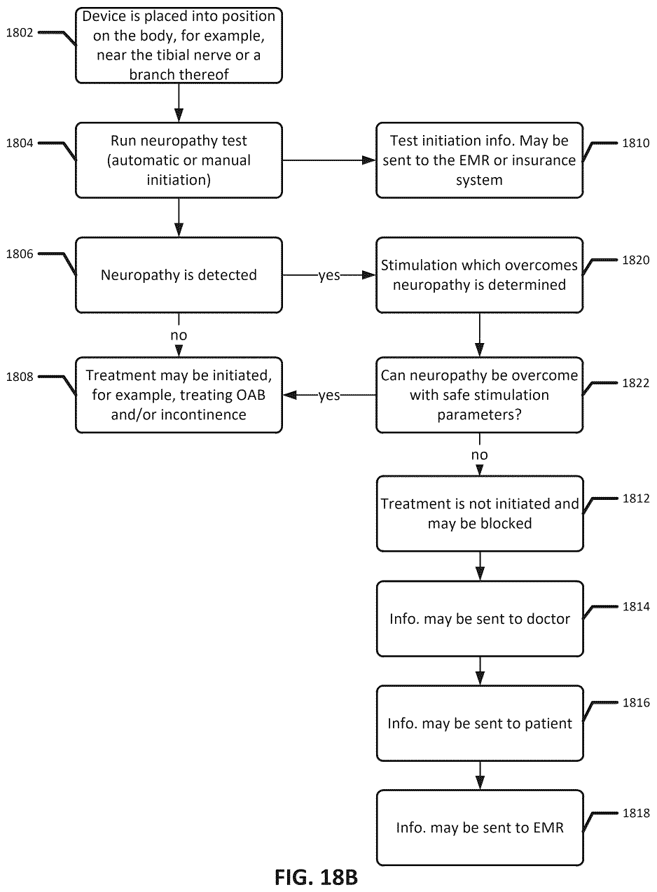

For example, the following steps (as shown in FIG. 18A) may be taken using the device:
Step 1802. The electrodes and sensor(s) for the device may be positioned on the user, for example as indicated in FIGS. 8 and 9. The electrodes and/or sensor may be placed in proximity to the tibial nerve or a branch thereof. The electrodes and/or sensor may be placed in proximity to the tibial nerve or a branch thereof on the ankle or foot. The electrodes may be transcutaneous or percutaneous. The sensor may be an EMG sensor. Alternatively, the electrodes and/or sensors may be placed in a location to stimulate and sense any nerve, for example, nerves other than the tibial nerve.
Step 1804. A neuropathy test may be performed using one or more of the electrodes and sensors (for example, EMG pads) in place. This test may be performed using F-wave velocity and/or latency and/or using any of the other parameters disclosed herein.
If neuropathy is not detected (step 1806), treatment may be initiated (step 1808). For example, the device may deliver stimulus to the tibial nerve or a branch thereof for treating overactive bladder and/or incontinence.
If neuropathy is detected (step 1806), treatment may be prevented (step 1812), for example, the controller may lock the device and prevent it from delivering any stimulus.
Step 1816. If neuropathy is detected, the controller may alert the user.
Step 1814 and step 1816. If neuropathy is detected, the controller may send a wireless communication to the user and/or the user's doctor.

In another example, the following steps (as shown in FIG. 18B) may be taken using the device:
Step 1802. The electrodes and sensor(s) for the device may be positioned on the user, for example as indicated in FIGS. 8 and 9. The electrodes and/or sensor may be placed in proximity to the tibial nerve or a branch thereof. The electrodes and/or sensor may be placed in proximity to the tibial nerve or a branch thereof on the ankle or foot. The electrodes may be transcutaneous or percutaneous. The sensor may be an EMG sensor. Alternatively, the electrodes and/or sensors may be placed in a location to stimulate and sense any nerve, for example, nerves other than the tibial nerve.
Step 1804. A neuropathy test may be performed using one or more of the electrodes and sensors (for example, EMG pads) in place. This test may be performed using F-wave velocity and/or latency and/or using any of the other parameters disclosed herein.
If neuropathy is not detected (step 1806), treatment may be initiated (step 1808). For example, the device may deliver stimulus to the tibial nerve or a branch thereof for treating overactive bladder and/or incontinence.
If neuropathy is detected (step 1806), the device may attempt to identify whether there is a safe stimulation which an overcome the neuropathy to initiate treatment. The system may attempt using different stimulation parameters, including different frequencies, amplitudes, waveforms, durations, stimulation patters etc. This is shown at step 1820.

If a safe stimulation can be found (step 1822), the treatment may be initiated. If a safe stimulation cannot be found, then treatment may be prevented (step 1812), for example, the controller may lock the device and prevent it from delivering any stimulus.
Step 1816. If neuropathy is detected, the controller may alert the user.
Step 1814 and step 1816. If neuropathy is detected, the controller may send a wireless communication to the user and/or the user's doctor.

The neuropathy test may be performed automatically by the device, or may be performed manually. The neuropathy test may be performed before every treatment or less frequently, for example it may be performed once/month, once every 6 months, or once/year.

To adequately assess the presence and/or absence of neuropathy, the neuropathy test may include one or more stimulus-sensing cycles. For example, the controller of the device may perform one, two, three, four or more stimulus/testing cycles to collect enough data to make an assessment of the existence and/or severity of neuropathy. The data may be averaged, filtered, or and/or otherwise manipulated by the controller. The cycles may be performed at one or more stimulation levels and frequencies. Where the stimulation cycle is repeated to collect enough data, the multiple cycles may be performed automatically in sequence.

In some embodiments, the performance and/or results of a neuropathy test are communicated in a wired or wireless manner to an electronic medical record (step 1818) and/or an insurance billing system (step 1810).

In some embodiments, the neuropathy test uses parameter collected from other users with and without neuropathy to determine the parameter thresholds for identifying neuropathy. In some embodiments, the neuropathy test uses parameter collected from the current user in either the same body location, or from different body locations to determine the parameter thresholds for identifying neuropathy.

In some embodiments, the device includes a neuropathy test button that the user and/or the user's doctor may press to run the neuropathy assessment test. In some embodiments, this test is performed by a doctor in his/her office. In some embodiments, this test is performed by the user at home or away from the doctor's office.

In some embodiments, the device may adjust the frequency, amplitude, duration and/or other parameter of the treatment based on the neuropathy detection results using neuropathy detection to dictate stimulation treatment.

In some embodiments, the location of neuropathy may be identified by placing the electrodes and/or sensors in different locations and running the neuropathy test.

In some embodiments, the stimulation required to overcome the neuropathy is identified. This may include stimulation parameters such as frequency, amplitude, waveform, pattern, etc.

In some embodiments, neuropathy testing may be performed using an electrode array, such as the electrode arrays shown in FIGS. 17A-17E . The electrode array may help with identifying the location of the neuropathy. The electrode array may help with identifying the stimulation amplitude, waveform, frequency, pattern etc. necessary to overcome the neuropathy

### Example of Data Processing System

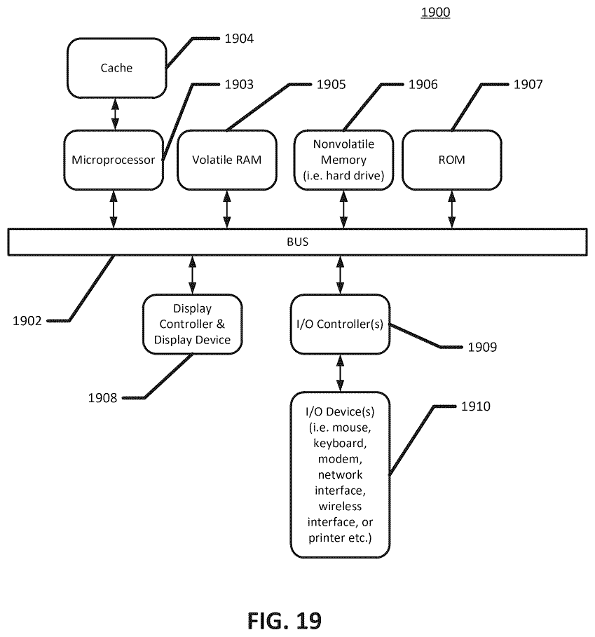

FIG. 19 is a block diagram of a data processing system, which may be used with any embodiment of the invention. For example, the system 1900 may be used as part of a controller, server, mobile device, hand piece, computer, tablet, etc. Note that while FIG. 19 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 19, the computer system 1900, which is a form of a data processing system, includes a bus or interconnect 1902 which is coupled to one or more microprocessors 1903 and a ROM 1907, a volatile RAM 1905, and a non-volatile memory 1906. The microprocessor 1903 is coupled to cache memory 1904. The bus 1902 interconnects these various components together and also interconnects these components 1903, 1907, 1905, and 1906 to a display controller and display device 1908, as well as to input/output (I/O) devices 1910, which may be mice, keyboards, modems, network interfaces, printers, and other devices which are well-known in the art.

Typically, the input/output devices 1910 are coupled to the system through input/output controllers 1909. The volatile RAM 1905 is typically implemented as dynamic RAM (DRAM) which requires power continuously in order to refresh or maintain the data in the memory. The non-volatile memory 1906 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 19 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 1902 may include one or more buses connected to each other through various bridges, controllers, and/or adapters, as is well-known in the art. In one embodiment, the I/O controller 1909 includes a USB (Universal Serial Bus) adapter for controlling USB peripherals. Alternatively, I/O controller 1909 may include an IEEE-1394 adapter, also known as FireWire adapter, for controlling FireWire devices.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the FIGS. can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the FIGS. herein may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Each of the individual variations described and illustrated herein has discrete components and features which may be readily separated from or combined with the features of any of the other variations. Modifications may be made to adapt a particular situation, material, composition of matter, process, process act(s) or step(s) to the objective(s), spirit or scope of the present invention.

Methods recited herein may be carried out in any order of the recited events which is logically possible, as well as the recited order of events. Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

What is claimed is:

1. An apparatus for performing electrical stimulation therapy, comprising:
an electrical stimulator configured for non-invasive placement against a first portion of the body such that the electrical stimulator is positionable into proximity to a target nerve within the first portion;
at least one sensor positioned along a second portion of the body; and
a controller in electrical communication with the electrical stimulator and the at least one sensor, wherein the controller is configured to:
deliver an electrical stimulus from the electrical stimulator to the target nerve;
detect electrical conduction through the target nerve or detect a muscular response caused by an electrical conduction through the target nerve via at least one sensor positioned along the second portion of the body;
receive a signal from the at least one sensor indicative of the detected electrical conduction or muscular response thereby providing feedback about the existence of neuropathic-irresponsiveness along the target nerve; and
adjust stimulation parameters of the electrical stimulator based on the feedback.

2. The apparatus of claim 1 wherein the controller is further configured to categorize a degree of the neuropathic-irresponsiveness into at least one stage of neuropathy.

3. The apparatus of claim 2 wherein the at least one stage of neuropathy comprises stage one, which is characterized by numbness and/or pain, stage two, which is characterized by constant pain, stage three, which is characterized by intense pain, or stage four, which is characterized by complete numbness and/or loss of sensation.

4. The apparatus of claim 1 wherein the controller is further configured to sense F-wave signals from the detected electrical conduction or muscular response, where the F-wave signals are indicative of the existence of neuropathic-irresponsiveness along the target nerve.

5. The apparatus of claim 1 wherein the controller is further configured to adjust the stimulation parameters based on a detection of neuropathic-irresponsiveness along the target nerve.

6. The apparatus of claim 1 wherein the controller is further configured to modify electrical stimulation therapy to the target nerve based on the detection of neuropathic-irresponsiveness.

7. The apparatus of claim 1 wherein the controller is further configured to modify an amplitude of the electrical stimulus to overcome the neuropathic-irresponsiveness.

8. The apparatus of claim 1 wherein the controller is further configured to modify a waveform of the electrical stimulus to overcome the neuropathic-irresponsiveness.

9. The apparatus of claim 1 wherein the controller is further configured to modify a frequency of the electrical stimulus to overcome the neuropathic-irresponsiveness.

10. The apparatus of claim 1 further comprising an array of electrodes in electrical communication with the electrical stimulator, wherein the array of electrodes is configured for non-invasive placement against the first portion of the body such that the array of electrodes is positionable into proximity to the target nerve within the first portion.

11. The apparatus of claim 1 wherein the controller is further configured to determine a location of neuropathic-irresponsiveness along the target nerve via the array of electrodes.

12. The apparatus of claim 11 wherein the controller is further configured to determine a location of the electrical stimulator to optimize detection of the electrical conduction or muscular response.

13. The apparatus of claim 1 wherein the controller is further configured to compare the signal from the at least one sensor against a range of values indicative of neuropathic-irresponsiveness.

14. The apparatus of claim 1 wherein the array of electrodes is configured for positioning into proximity of a sciatic, tibial, or ulnar nerve, or a branch thereof, within the first portion of the body.

15. A method of performing electrical stimulation therapy, comprising:
non-invasively positioning a first portion of a patient's body relative to an electrical stimulator such that a target nerve within the first portion of the body is in proximity to the electrical stimulator;
delivering an electrical stimulus from the electrical stimulator to the target nerve;
detecting electrical conduction through a response nerve or detecting a muscular response caused by an electrical conduction through the response nerve via at least one sensor positioned along a second portion of the body;
receiving a signal from the at least one sensor indicative of the detected electrical conduction or muscular response thereby providing feedback about the existence of neuropathic-irresponsiveness along the target nerve; and
adjusting stimulation parameters of the electrical stimulator based on the feedback.

16. The method of claim 15 wherein the target nerve and the response nerve are the same.

17. The method of claim 15 wherein the first portion of the body and the second portion of the body are the same.

18. The method of claim 15 further comprising categorizing a degree of the neuropathic-irresponsiveness into at least one stage of neuropathy.

19. The method of claim 18 wherein the at least one stage of neuropathy comprises stage one, which is characterized by numbness and/or pain, stage two, which is characterized by constant pain, stage three, which is characterized by intense pain, or stage four, which is characterized by complete numbness and/or loss of sensation.

20. The method of claim 15 wherein receiving the signal from the at least one sensor comprises sensing F-wave signals from the detected electrical conduction or muscular response, where the F-wave signals are indicative of the existence of neuropathic-irresponsiveness along the target nerve.

21. The method of claim 15 wherein adjusting stimulation parameters of the electrical stimulator comprises adjusting a modulation of the electrical stimulus.

22. The method of claim 15 wherein adjusting stimulation parameters of the electrical stimulator comprises adjusting a frequency of the electrical stimulus.

23. The method of claim 15 wherein adjusting stimulation parameters of the electrical stimulator comprises adjusting a waveform of the electrical stimulus.

24. The method of claim 15 wherein adjusting stimulation parameters of the electrical stimulator comprises adjusting at least one of modulation, frequency, or waveform of the electrical stimulus.

25. The method of claim 15 wherein adjusting stimulation parameters of the electrical stimulator comprises adjusting at least one of modulation, frequency, and waveform of the electrical stimulus.

26. The method of claim 15 wherein adjusting stimulation parameters of the electrical stimulator comprises adjusting a position of the electrical stimulator relative to the first portion of the body.

27. The method of claim 15 wherein adjusting stimulation parameters of the electrical stimulator comprises adjusting at least one of modulation, frequency, waveform, and positioning of the electrical stimulator.

28. The method of claim 15 further comprising determining a location of neuropathic-irresponsiveness along the response nerve.

29. The method of claim 28 wherein determining the location of neuropathic-irresponsiveness comprises delivering the stimulus or receiving the signal via an array of electrodes configured for placement against the first portion of the body.

30. The method of claim 15 wherein delivering the electrical stimulus comprises delivering the stimulus or delivering the electrical stimulation via an array of electrodes spatially configured for placement against the first portion of the body.

31. The method of claim 30 wherein the array of electrodes is configured to deliver the electrical stimulation intermittently to determine a location for positioning of the electrical stimulator relative to the first portion of the body.

32. The method of claim 15 wherein providing feedback about the existence of neuropathic-irresponsiveness comprises comparing the signal from the at least one sensor against a range of values indicative of neuropathic-irresponsiveness.

33. The method of claim 32 wherein the range of values is obtained from a patient population.

34. The method of claim 32 wherein the range of values is obtained from the patient.

35. The method of claim 15 further comprising applying the electrical stimulation therapy to mitigate incontinence in the patient.

36. The method of claim 15 further comprising applying the electrical stimulation therapy to mitigate over active bladder in the patient.

37. The method of claim 15 further comprising applying the electrical stimulation therapy to mitigate menstrual pains, to mitigate substance abuse, tremors, or Parkinsons in the patient.

38. The method of claim 15 wherein non-invasively positioning comprises positioning the first portion of the patient's body relative to the electrical stimulator such that a sciatic nerve, or a branch thereof, within the first portion of the body is in proximity to the electrical stimulator.

39. The method of claim 15 wherein non-invasively positioning comprises positioning the first portion of the patient's body relative to the electrical stimulator such that a tibial nerve, or a branch thereof, within the first portion of the body is in proximity to the electrical stimulator.

40. The method of claim 15 wherein non-invasively positioning comprises positioning the first portion of the patient's body relative to the electrical stimulator such that an ulnar nerve, or a branch thereof, within the first portion of the body is in proximity to the electrical stimulator.
